(19) United States
(12) Patent Application Publication
(10) Pub. No.: US 2005/0003347 A1
(43) Pub. Date: Jan. 6, 2005

(54) Domain-exchanged binding molecules, methods of use and methods of production

(76) Inventors: Daniel Calarese, San Diego, CA (US); Dennis Burton, La Jolla, CA (US); Ian Wilson, La Jolla, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/838,153

(22) Filed: Apr. 30, 2004

Related U.S. Application Data
(60) Provisional application No. 60/468,503, filed on May 6, 2003.

(51) Int. Cl. C12Q 1/70; C07K 16/10

(52) U.S. Cl. 435/5; 530/388.3

(57) ABSTRACT

Methods for random or rational design of high affinity domain exchanged binding molecules and methods of use are provided herein. Also provided are libraries containing a plurality of such domain exchanged binding molecules.

### RELATED APPLICATIONS

This application claims priority under 35 U.S.C. §119(e) to U.S. Provisional Application Ser. No. 60/468,503, filed May 6, 2003, which is herein incorporated by reference in its entirety.

### GRANT INFORMATION

The present invention was made with support from a grant from the National Institutes of Health Grant Nos. GM46192 and AI33292. The government may have certain rights in the invention.

### FIELD OF THE INVENTION

The present invention relates generally to the field of immunology and specifically to domain-exchanged binding molecules with unique binding properties.

### BACKGROUND OF THE INVENTION

Carbohydrates are present at the surface of bacterial cell envelopes either as capsular polysaccharides or as lipopolysaccharides when linked to a lipid. These surface polysaccharides can be the basis for serogroup and serotype classification amongst the various bacterial families, act as bacterial virulence factors, and are major targets of the host's immune response upon infection. Protective immune responses against microbial pathogens are frequently based on anti-carbohydrate antibodies produced against polysaccharides located on their cell surface. Because many bacterial polysaccharides are immunogenic, the potential use of polysaccharides in antibacterial vaccination is an area of increasing scientific interest.

Altered glycosylation in host cells associated with viral infection has been reported (Ray et al. (1978) Virology 88:118; Kumarasamy et al. (1985) Arch. Biochem. Biphys. 236:593). Like oncogenesis, aberrant glycosylation induced by cytomegalovirus or by HIV causes formation of new antigens which are absent in the original host cells (Andrews et al. (1989) J. Exp. Med. 169:1347; Adachi et al. (1988) J. Exp. Med. 167:323).

There is also mounting evidence suggesting that immunization-based strategies can be used to mobilize the immune system against specific carbohydrate antigens displayed on the surface of cancer cells. The level of expression of cell surface carbohydrate antigens is often significantly increased on carcinogenic transformation, and, in some cases, the expression of particular antigens appears to be associated primarily with the transformed state. Thus, carbohydrate-specific antibodies offer the potential for a targeted immunotherapeutic approach to the treatment of certain forms of cancer, as well as the identification carbohydrate containing-antigens for use in immunization.

Thus, strategies for identifying immunotherapeutic agents that bind to complex antigens that include repeating units, for example, carbohydrates, are desirable.

### SUMMARY OF THE INVENTION

The present invention is based on the seminal discovery of the structure of unique domain-exchanged binding molecules having increased affinity and greater avidity for antigens arrayed on a surface, and typically antigens having repeating units, such as carbohydrates. The invention domain-exchanged binding molecules have unique structures and binding characteristics and are capable of binding to different types of antigens with affinities not previously achieved.

The invention provides domain-exchanged binding molecules comprising a heavy chain with a variable region and optionally a constant region and a multivalent binding surface comprising two conventional antigen binding sites and at least one non-conventional binding site formed by an interface between adjacently positioned heavy chain variable regions; with the proviso that the molecule is not a conventional 2G12 antibody. Depending on the structural conformation of the particular domain-exchanged binding molecule, the conventional sites, non-conventional site(s) or a combination of both may be utilized for binding a particular antigen. Invention molecules also include a non-naturally occurring (e.g., synthetic) domain-exchanged binding molecule comprising a heavy chain with a variable region and a constant region and a multivalent binding surface comprising two conventional antigen binding sites and at least one non-conventional binding site formed by an interface between adjacently positioned heavy chain variable regions.

In one aspect, the invention provides a method of producing a domain-exchanged binding molecule having affinity for repeating units, or epitopes, such as carbohydrates. The method allows for production of such molecules capable of binding an antigen by providing a library of molecules that are randomly generated. In such libraries, the antibody combining site may be randomized to provide a plurality of binding molecules with different antigen specificity, for example, while maintaining a framework of at least V_{L}-V_{H}-V_{H}-V_{L} similar to the 2G12 antibody described herein.

In another aspect, production of invention domain-exchanged binding molecules is by rational design, for example of existing conventional antibody structures, such as anti-HIV or anti-CD20 antibodies.

In one embodiment, the invention provides method of treating a subject having or at risk of having an infection or disease by a pathogen or agent containing repeating units on its surface, such as a viral coat or envelope, bacterial membranes, or the like. Such a method can be performed, for example, by administering to the subject a therapeutically effective amount of a domain-exchanged binding molecule of the invention that binds to the pathogen or agent, thereby providing passive immunization to the subject. Such a method can be useful as a prophylactic method, thus reducing the likelihood that a subject can become infected with the pathogen or agent, or as a therapeutic for a subject infected with the pathogen or agent.

In another embodiment, the invention provides diagnostic assays utilizing domain-exchanged binding molecules of the invention, rather than using conventional antibodies. Such assays can be any immunoassay for which conventional antibodies are typically utilized, however, the binding molecules of the invention may provide increased sensitivity for particular antigens, as compared with conventional antibodies. Invention binding molecules can be used in combination with conventional antibodies as well for immunoassays.

### BRIEF DESCRIPTION OF THE DRAWINGS

FIG. 1A-D illustrate the novel architecture of antibody 2G12 and structural factors that promote the Fab V_{H}/V_{H}′ domain exchange. Figures were generated using programs Bobscript (67), Molscript(68), and Raster3D (69).

FIG. 1A illustrates the monomer of Fab 2G12 in the crystal showing that the V_{H} clearly separates from its normal interaction with the V_{L}. The light and heavy chains are shown in cyan and red, respectively. The monomer does not exist in the crystal, but only in the context of the domain-swapped dimer.

FIG. 1B shows the structure of the two domain-swapped Fab molecules, as they assemble in the crystal. Both light chains are shown in cyan, with the heavy chains from Fab 1 and Fab 2 shown in red and purple. The distance between the two conventional combining sites is indicated.

FIG. 1C illustrates a close up view in ball-and-stick representation of the novel V_{H}/V_{H}′ interface between the variable heavy domains. Potential hydrogen bonds are shown with dashed black lines.

FIG. 1D shows the elbow region between the constant heavy and variable heavy domains and illustrates the domain exchange. The linker region between V_{H}′ and C_{H}1 is shown in ball and stick with corresponding 2Fo-Fc electron density contoured at 1.5σ.

FIGS. 2A and 2B illustrate biophysical evidence for a domain-exchanged dimer of 2G12 in solution.

FIG. 2A shows gel filtration of Fab 2G12 and b12 from papain digests.

Retention times are indicated on the x-axis, and protein concentration on the y-axis as measured by UV absorbance.

FIG. 2B illustrates sedimentation coefficients of IgG1 2G12 relative to other IgG1 molecules (b6, b12, and 2F5, all anti-HIV-1 antibodies). The x-axis indicates the range of s_{20,W} values and the y-axis is the relative concentration (measured by UV absorbance) of the protein at that point.

FIG. 3A-C illustrates interactions of the Fab 2G12 dimer with Man₉GlcNAc₂. Figures were generated using programs Bobscript, Molscript, and Raster3D.

FIG. 3A shows the chemical structure of Man₉GlcNAc₂. Red sugars make contacts with Fab 2G12 at the primary binding site (conventional combining pocket), while blue sugars contact Fab 2G12 at the secondary binding site (the unusual V_{H}/V_{H}′ interface).

FIG. 3B is a ball-and-stick representation of Man₉GlcNAc₂ bound to the primary binding site of Fab 2G12, with corresponding 2Fo-Fc electron density contoured at 1.6σ.

FIG. 3C illustrates the overall structure of the Fab 2G12 dimer bound to Man₉GlcNAc₂ in two orthogonal views. A total of four Man₉GlcNAc₂ moieties are bound to each Fab dimer. The red sugars of the Man₉GlcNAc₂ moieties (corresponding to FIG. 3A) are bound in the primary binding site, and the blue sugars of the Man₉GlcNAc₂ moieties are bound at the secondary V_{H}/V_{H}′ interface.

FIG. 4A-C illustrate the antibody combining site interactions with the disaccharide Manα1-2Man. The Figures were generated using programs Bobscript, Molscript, Raster3D, and GRASP (72).

FIG. 4A shows the 2Fo-Fc electron density for Manα1-2Man is contoured at 1.7σ and the CDR loops are labelled.

FIG. 4B illustrates the molecular surface of Fab 2G12 at the primary binding site of Manα1-2Man. Molecular surface from CDR's L3, H1, H2, and H3 are colored in cyan, green, blue, and purple, respectively.

FIG. 4C is a ball-and-stick figure of the combining site showing Fab atoms within hydrogen bonding distance of Manα1-2Man (dotted lines). The Fab heavy chain and light chain are shown in purple and cyan, respectively.

FIG. 5 shows results of inhibition of 2G12 binding to HIV-1 gp120, with IC₅₀ values of different carbohydrates relative to the IC₅₀ value of mannose.

FIG. 6 illustrates alanine scanning mutagenesis of Fab 2G12, with the relative apparent binding affinities of Fab 2G12 mutants being indicated on the structure. Results are shown relative to wild type Fab 2G12 binding of gp120_{JR-FL}(100%). Residues that are black indicate that an alanine substitution at that position resulted in no significant effect (50% to 200% relative to wild type) on apparent binding affinity of 2G12 for gp120_{JR-FL}, while residues in red (labeled) indicate an alanine substitution at that position resulted in a significant (>2-fold) decrease in apparent binding affinity of 2G12 for gp120_{JR-FL}. The Figure was generated using programs Molscript and Raster3D.

FIG. 7 shows a model of the domain-exchanged Fab dimer of 2G12 interacting with gp120. The cluster of five glycosylation sites on gp120 that have previously been implicated (13) in 2G12 binding are indicated in red and labeled (asparagines at positions 295, 332, 339, 386, and 392). Three separate Man₉GlcNAc₂ moieties, shown in blue (two in the primary combining sites and one in the V_{H}/V_{H}′ interface), can easily be accommodated without any major rearrangements of either the Asn residues or the bound carbohydrates of 2G12. In this model, the carbohydrates at the primary combining sites originate from Asn 332 and Asn 392 in gp120, whereas the carbohydrate located at the V_{H}/V_{H}′ interface would arise from Asn 339. The Man₉GlcNAc₂ moeities interacting with the primary combining sites are unaltered from those in the 2G12-Man₉GlcNAc₂ crystal structure and can easily be connected to Asn 332 and Asn 392 on gp120. For the V_{H}/V_{H}′ interface carbohydrate, only the two distal N-acetyl glucosamine rings are adjusted to model this interaction. Other combinations or permutations of these closely-packed carbohydrates occupying the primary and secondary binding sites are possible. The Figure was generated using programs Molscript and Raster3D.

FIG. 8 shows a stereo view of the twist between the variable and constant domains of Fab 2G12. Fab 2G12 is shown in blue, while a “typical” Fab (Fab 1dba, PDB code DB3) is shown in grey. The light chain is on the left, and the heavy chain is on the right. Residues 114 to 200 of the light chain constant domains of Fab 2G12 were aligned with a library of 172 Fab molecules. To show the relationship between the variable and constant domains, the positions of residue L107 in Fab 2G12 and the library of Fabs is shown (yellow dots). The corresponding position in the heavy chain, residue H113, was then plotted for the library of all molecules (cyan dots) and 2G12 (red dot). The library of Fab molecules all have similar arrangements of their variable and constant domains (as represented by the tight cluster of cyan dots relative to the “fixed” yellow dots), while the Fab 2G12 variable domain is highly twisted (red dot) relative to its constant domain. The Figure was made using Molscript and rendered with Raster3D.

FIG. 9 illustrates the missing ball-and-socket interaction between V_{H} and C_{H}1 domains. Light chain is shown in cyan, while the two heavy chains in the Fab dimer are shown in red and purple. Phe^{H146} normally serves as the “ball”, fitting into a “socket” made by residues Leu^{H11}, Thr^{H110}, and Ser^{H112}. The Figure was made using Molscript and rendered with Raster3D.

FIG. 10A-B show the results of sedimentation equilibrium of Fab 2G12 and NC-1. FIG. 10A shows control Fab NC-1, which runs as a one species monomer. FIG. 10B shows Fab 2G12, with a two species fit. These species correspond to the molecular weights of Fab monomers and dimers (which are 45.7 kD and 95.7 kD, respectively).

FIG. 11 illustrates a stereo view of the interactions of Fab 2G12 dimer bound to Man₉GlcNAc₂ residues. Red sugars make contacts with Fab 2G12 at the primary binding site (conventional combining pocket), while blue sugars contact Fab 2G12 at the secondary binding site (the unusual V_{H}/V_{H}′ interface). The Figure was made using Molscript and rendered with Raster3D.

FIG. 12 shows three structural characteristics of 2G12 Fabs in domain exchange, also applicable to other V_{L}-V_{H}-V_{H}-V_{L} containing binding molecules of the invention.

Table 1 provides a summary of crystallographic data. Crystals of the unliganded Fab and the Fab bound to disaccharide Manα1-2Man exhibit mildly anisotropic diffraction, while the crystals of Fab 2G12 bound to oligosaccharide Man₉GlcNAc₂ show strong anisotropic diffraction. This property is reflected in the overall anisotropic B-values of each crystal. However, the electron density maps are clearly interpretable. Constant domains generally have higher B values relative to the variable domains. ^{a}Numbers in parenthesis are for highest resolution shell. ^{b}All Wilson B values are calculated from 4.0 Å to the highest resolution of that data set. ^{c}Calculated using PROCHECK (73). ^{d}Includes residue L51 of both Fab molecules in the asymmetric unit, which exists in a γ turn, but is flagged by PROCHECK as an outlier. Other residues designated as disallowed by PROCHECK have a good fit to the corresponding electron density.

Table 2 presents relative apparent binding affinities of Fab 2G12 mutants. Results are shown relative to wild type Fab 2G12 binding (100%). Mutations occuring at the V_{H}/V_{H}′ interface, primary combining site, or the secondary (V_{H}/V_{H}′ interface) binding site are indicated.

### DETAILED DESCRIPTION OF THE INVENTION

The present invention is based on the seminal discovery of a domain exchanged binding molecule that interlocks heavy chain variable regions of an immunoglobulin molecule to provide at least one non-conventional binding site. A heavy chain V_{H} exchanges (domain-swaps or exchanges) with a second V_{H} region, so that the first V_{H} region interacts with the opposite V_{L}, and optionally with the opposite C_{H1} and C_{L}. This arrangement is formed from two intertwined parallel side-by-side regions and creates a multivalent binding site composed of the two conventional antigen binding sites and at least one non-conventional site formed from a V_{H}-V_{H} interface that could act as a third or fourth antigen binding region. It is possible that the V_{H}-V_{H} interface could provide one or two antigen binding sites and the conventional binding sites might not bind antigen. One illustrative example of an invention domain exchanged binding molecule includes a V_{L}-V_{H}-V_{H}-V_{L} including an entire Fab region.

Such domain-exchanged binding molecules of the invention have enhanced affinities that would be relevant for weak or poor antigens including those with repeating units, for example, carbohydrates, where the maximum monovalent binding is often only in the micromolar range, as well as for other antigens. Such a grouping of binding sites could lead to a greater avidity for antigens arrayed on a surface, such as a viral coat, bacterial membrane, tumor cell or some artificial array. The combined binding surface may then have novel properties for binding antigens. The 2G12 anti-HIV-1 antibody described herein shown merely as an illustrative example of a domain-exchanged binding molecule. The crystal structure of 2G12 indicated that the molecule was produced by substituting about four residues in the V_{H} and elbow region of an immunoglobulin. However, a domain exchanged binding molecule of the invention may include as few as one amino acid residue change to provide a structural conformation resulting in a V_{L}-V_{H}-V_{H}-V_{L} molecule as described herein.

The following includes some relevant definitions.

Amino Acid Residue: An amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxy terminus of a polypeptide. In keeping with standard polypeptide nomenclature (described in J. Biol. Chem., 243:3552-59 (1969) and adopted at 37 CFR 1.822(b)(2)), abbreviations for amino acid residues are shown in the following

Table of Correspondence:

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left-to-right orientation in the conventional direction of amino terminus to carboxy terminus. In addition, the phrase “amino acid residue” is broadly defined to include the amino acids listed in the Table of Correspondence and modified and unusual amino acids, such as those listed in 37 CFR 1.822(b)(4), and incorporated herein by reference. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Recombinant DNA (rDNA) molecule: A DNA molecule produced by operatively linking two DNA segments. Thus, a recombinant DNA molecule is a hybrid DNA molecule comprising at least two nucleotide sequences not normally found together in nature. rDNA's not having a common biological origin, i.e., evolutionarily different, are said to be “heterologous.”

Vector: A rDNA molecule capable of autonomous replication in a cell and to which a DNA segment, e.g., gene or polynucleotide, can be operatively linked so as to bring about replication of the attached segment. Vectors capable of directing the expression of genes encoding for one or more polypeptides are referred to herein as “expression vectors”. Particularly important vectors allow cloning of cDNA (complementary DNA) from mRNAs produced using reverse transcriptase. An expression vector (or the polynucleotide) generally contains or encodes a promoter sequence, which can provide constitutive or, if desired, inducible or tissue specific or developmental stage specific expression of the encoding polynucleotide, a poly A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37 42, 1993; Kirshenbaum et al., J. Clin. Invest. 92:381-387, 1993; each of which is incorporated herein by reference).

Isolated: The term isolated is used herein to refer to altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

Antibody: The term antibody in its various grammatical forms is used herein to refer to immunoglobulin molecules and immunologically active portions of immunoglobulin molecules, i.e., molecules that contain an antibody combining site or paratope. Exemplary antibody molecules are intact immunoglobulin molecules, substantially intact immunoglobulin molecules and portions of an immunoglobulin molecule, including those portions known in the art as Fab, Fab′, F(ab′)2 and F(v). The term “antibody” includes naturally occurring antibodies as well as non naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275 1281 (1989), which is incorporated herein by reference). These and other methods of making, for example, chimeric, humanized, CDR grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference)

Domain exchanged binding molecules of the invention include single chain molecules as well as molecules that do not contain constant regions, for example, V_{L-}V_{H-}V_{H-}V_{L} molecules either with our without a dimerization domain. Thus, a minimal structure of the invention is the V_{L-}V_{H-}V_{H-}V_{L} structure with no constant region.

Antibody Combining Site: An antibody combining site in a conventional antibody is that structural portion of an antibody molecule comprised of a heavy and light chain variable and hypervariable regions that specifically binds (immunoreacts with) an antigen. The term immunoreact in its various forms means specific binding between an antigenic determinant-containing molecule and a molecule containing an antibody combining site such as a whole antibody molecule or a portion thereof. As discussed below, an antibody combining site can also be formed by a V_{H}-V_{H} interface in the domain exchanged binding molecules of the invention.

The term “HIV-induced disease” means any disease caused, directly or indirectly, by HIV. An example of a HIV-induced disease is acquired autoimmunodeficiency syndrome (AIDS), and any of the numerous conditions associated generally with AIDS which are caused by HIV infection.

The term “conventional binding site” or “region” refers to traditional Fab region on an immunoglobulin molecule having a “variable” region of the heavy and the light chain to provide specificity for binding an epitope or antigen. The standard “Y” shaped antibody molecule contains two regions with two antibody binding sites, referred to herein as “conventional” binding sites. A minimal binding molecule of the invention does not require an intact Fab, as long as the structure includes at least V_{L}-V_{H}-V_{H}-V_{L}.

The term “non-conventional binding site” or “region” or “unconventional binding site” or “region” refers to the exchanged or swapped heavy chain regions of the variable domain of the Fab of a traditional immunoglobulin molecule which form a novel binding site or region. This region is also referred to herein as the V_{H}-V_{H} interface. Domain exchanged binding molecules of the invention are characterized as having conventional and non-conventional binding sites or regions and a minimal structure of V_{L}-V_{H}-V_{H}-V_{L}.

The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. The term “conservative variation” also includes the use of a substituted amino acid in place of an unsubstituted parent amino acid provided that antibodies having the substituted polypeptide also neutralize HIV. Analogously, another preferred embodiment of the invention relates to polynucleotides which encode the above noted heavy and/or light chain polypeptides and to polynucleotide sequences which are complementary to these polynucleotide sequences. Complementary polynucleotide sequences include those sequences which hybridize to the polynucleotide sequences of the invention under stringent hybridization conditions.

The present invention contemplates therapeutic compositions useful for practicing the therapeutic methods described herein. Therapeutic compositions of the present invention contain a physiologically tolerable carrier together with at least one species of domain exchanged binding molecules as described herein, dissolved or dispersed therein as an active ingredient.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a subject such as a human without the production of undesirable physiological effects such as nausea, dizziness, gastric upset and the like.

The preparation of a pharmacological composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes.

Liquid compositions can also contain liquid phases in addition to and to the exclusion of water. Exemplary of such additional liquid phases are glycerin, vegetable oils such as cottonseed oil, organic esters such as ethyl oleate, and water-oil emulsions.

A representative subject for practicing passive immunotherapeutic methods is any human exhibiting symptoms of HIV-induced disease, including AIDS or related conditions believed to be caused by HIV infection, and humans at risk of HIV infection. Patients at risk of infection by HIV include babies of HIV-infected pregnant mothers, recipients of transfusions known to contain HIV, users of HIV contaminated needles, individuals who have participated in high risk sexual activities with known HIV-infected individuals, and the like risk situations.

In addition to primates, such as humans, a variety of other mammals can be treated according to the methods of the present invention. For instance, mammals including, but not limited to, cows, sheep, goats, horses, dogs, cats, guinea pigs, rats or other bovine, ovine, equine, canine, feline, rodent or murine species can be treated. The method can also be practiced in other species, such as avian species (e.g., chickens).

The dosage of a domain exchanged binding molecule can be adjusted by the individual physician in the event of any complication. A therapeutically effective amount of domain exchanged binding molecule of this invention is typically an amount of domain exchanged binding molecule such that when administered in a physiologically tolerable composition is sufficient to achieve a plasma concentration of from about 0.1 microgram (μg) per milliliter (ml) to about 100 μg/ml, preferably from about 1 μg/ml to about 5 μg/ml, and usually about 5 μg/ml. Stated differently, the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily, for one or several days.

The domain exchanged binding molecules of the invention can be administered parenterally by injection or by gradual infusion over time. Thus, domain exchanged binding molecules of the invention can be administered intravenously, intraperitoneally, intramuscularly, subcutaneously, intracavity, transdermally, and can be delivered by peristaltic means, for example.

The therapeutic compositions of this invention are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle.

The therapeutic compositions may be administered by any suitable means, for example, orally, such as in the form of tablets, capsules, granules or powders; sublingually; buccally; parenterally, such as by subcutaneous, intravenous, intramuscular, intrathecal, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents. The present compounds may, for example, be administered in a form suitable for immediate release or extended release. Immediate release or extended release may be achieved by the use of suitable pharmaceutical compositions comprising the present compounds, or, particularly in the case of extended release, by the use of devices such as subcutaneous implants or osmotic pumps. The present compounds may also be administered liposomally.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The invention also relates to a method for preparing a medicament or pharmaceutical composition comprising the domain exchanged binding molecules of the invention. The medicament is useful for the treatment of infections or diseases, e.g., a tumor, where it is desirable to have a binding molecule that has high affinity and high avidity for an antigen, especially those having repeating units, such as carbohydrates.

Domain-exchanged binding molecules used in the method of the invention are suited for use, for example, in immunoassays in which they can be utilized in liquid phase or bound to a solid phase carrier. In addition, the domain-exchanged binding molecules in these immunoassays can be detectably labeled in various ways. Examples of types of immunoassays which can utilize domain-exchanged binding molecules of the invention are competitive and non-competitive immunoassays in either a direct or indirect format. Examples of such immunoassays are the radioimmunoassay (RIA) and the sandwich (immunometric) assay. Detection of the antigens using the domain-exchanged binding molecules of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The term “immunometric assay” or “sandwich immunoassay”, includes simultaneous sandwich, forward sandwich and reverse sandwich immunoassays. These terms are well understood by those skilled in the art. Those of skill will also appreciate that domain-exchanged binding molecules according to the present invention will be useful in other variations and forms of assays which are presently known or which may be developed in the future. These are intended to be included within the scope of the present invention.

As disclosed herein, the invention provides an advantage that certain aspects can be adapted to high throughput analysis. For example, combinatorial libraries of domain-exchanged binding molecules can be screened in order to identify molecules that bind to a specific pathogen, agent, or molecule, typically containing repeating units on its surface. Alternatively, in adapting the methods of the invention to a high throughput format, a biological sample (e.g., test cells, or extracts of test cells), can be arranged in an array, which can be an addressable array, on a solid support such as a microchip, a glass slide, or a bead, and cells (or extracts) can be contacted serially or in parallel with one or more domain-exchanged binding molecules as disclosed herein. Samples arranged in an array or other reproducible pattern can be assigned an address (i.e., a position on the array), thus facilitating identification of the source of the sample. An additional advantage of arranging the samples in an array, particularly an addressable array, is that an automated system can be used for adding or removing reagents from one or more of the samples at various times, or for adding different reagents to particular samples. In addition to the convenience of examining multiple samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays. Conveniently, cells or extracts at a position in the array can be contacted with two or more domain-exchanged binding molecules (e.g., additional antibodies), wherein the domain-exchanged binding molecules are differentially labeled or comprise a reaction that generates distinguishable products, thus providing a means for performing a multiplex assay. Such assays can allow the examination of one or more, particularly 2, 3, 4, 5, 10, 15, 20, or more pathogens, agents, or molecules containing repeating units on their surfaces to identify subjects having or at risk of having infection or disease.

There are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. Those of ordinary skill in the art will know of other suitable labels for binding to the domain-exchanged binding molecules, or will be able to ascertain such, using routine experimentation. Another technique which may also result in greater sensitivity consists of coupling the domain-exchanged binding molecules to low molecular weight haptens. These haptens can then be specifically detected by means of a second reaction. For example, it is common to use such haptens as biotin, which reacts with avidin, or dinitrophenyl, puridoxal, and fluorescein, which can react with specific antihapten antibodies.

Domain-exchanged binding molecules can be bound to many different carriers and used to detect the presence of antigen in a biological sample. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding domain-exchanged binding molecules, or will be able to ascertain such using routine experimentation.

The biological samples may be obtained from any bodily fluids, for example, blood, urine, saliva, phlegm, gastric juices, cultured cells, biopsies, or other tissue preparations (e.g., tumor cells).

In performing the assays it may be desirable to include certain “blockers” or “blocking agents” in the incubation medium (usually added with the labeled soluble antibody). The “blockers” or “blocking agents” are added to assure that non-specific proteins, proteases, or anti-heterophilic immunoglobulins to anti-immunoglobulins present in the experimental sample do not cross-link or destroy the antibodies on the solid phase support, or the radiolabeled indicator antibody, to yield false positive or false negative results. The selection of “blockers” or “blocking agents” therefore may add substantially to the specificity of the assays described in the present invention.

It has been found that a number of nonrelevant (i.e., nonspecific) antibodies of the same class or subclass (isotype) as those used in the assays (e.g., IgG1, IgG2a, IgM, etc.) can be used as “blockers” or “blocking agents.” The concentration of the “blockers” (normally 1-100 μg/μl) may be important, in order to maintain the proper sensitivity yet inhibit any unwanted interference by mutually occurring cross reactive proteins in the specimen.

In using a domain-exchanged binding molecule for the in vivo detection of antigens, the detectably labeled domain-exchanged binding molecule is given in a dose which is diagnostically effective. The term “diagnostically effective” means that the amount of detectably labeled domain-exchanged binding molecule is administered in sufficient quantity to enable detection of the site having the antigen for which the domain-exchanged binding molecules are specific. The concentration of detectably labeled domain-exchanged binding molecule which is administered should be sufficient such that the binding to those cells having antigen is detectable compared to the background. Further, it is desirable that the detectably labeled domain-exchanged binding molecule be rapidly cleared from the circulatory system in order to give the best target-to-background signal ratio.

As a rule, the dosage of detectably labeled domain-exchanged binding molecule for in vivo diagnosis will vary depending on such factors as age, sex, and extent of disease of the individual. The dosage of domain-exchanged binding molecule can vary from about 0.001 mg/m² to about 500 mg/m², preferably 0.1 mg/m² to about 200 mg/m², most preferably about 0.1 mg/m² to about 10 mg/m². Such dosages may vary, for example, depending on whether multiple injections are given, tumor burden, and other factors known to those of skill in the art.

For in vivo diagnostic imaging, the type of detection instrument available is a major factor in selecting a given radioisotope. The radioisotope chosen must have a type of decay which is detectable for a given type of instrument. Still another important factor in selecting a radioisotope for in vivo diagnosis is that the half-life of the radioisotope be long enough so that it is still detectable at the time of maximum uptake by the target, but short enough so that deleterious radiation with respect to the host is minimized. Ideally, a radioisotope used for in vivo imaging will lack a particle emission, but produce a large number of photons in the 140-250 keV range, which may be readily detected by conventional gamma cameras.

For in vivo diagnosis, radioisotopes may be bound to immunoglobulin either directly or indirectly by using an intermediate functional group. Intermediate functional groups which often are used to bind radioisotopes which exist as metallic ions to immunoglobulins are the bifunctional chelating agents such as diethylenetriaminepentacetic acid (DTPA) and ethylenediaminetetraacetic acid (EDTA) and similar molecules. Typical examples of metallic ions which can be bound to the domain-exchanged binding molecules of the invention are ¹¹¹In, ⁹⁷Ru, ⁶⁷Ga, ⁶⁸Ga, ⁷²As, ⁸⁹Zr, and ²⁰¹T1.

A domain-exchanged binding molecule useful in the method of the invention can also be labeled with a paramagnetic isotope for purposes of in vivo diagnosis, as in magnetic resonance imaging (MRI) or electron spin resonance (ESR). In general, any conventional method for visualizing diagnostic imaging can be utilized. Usually gamma and positron emitting radioisotopes are used for camera imaging and paramagnetic isotopes for MRI. Elements which are particularly useful in such techniques include ¹⁵⁷Gd, ⁵⁵Mn, ¹⁶²Dy, ⁵²Cr, and ⁵⁶Fe.

The present invention also describes a diagnostic system, preferably in kit form, for assaying for the presence of an antigen, e.g., a pathogen, bacteria, virus, tumor, in a sample according to the diagnostic methods described herein. A diagnostic system includes, in an amount sufficient to perform at least one assay, at least one domain exchanged binding molecule of the invention alone or in combination with a traditional antibody, as a separately packaged reagent.

“Instructions for use” typically include a tangible expression describing the reagent concentration or at least one assay method parameter such as the relative amounts of reagent and sample to be admixed, maintenance time periods for reagent/sample admixtures, temperature, buffer conditions and the like.

Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

With respect to cancer, the presence of a relatively high amount of antigen or similar molecule in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

The present invention describes methods for producing novel domain exchanged binding molecules. The methods are based generally on the use of combinatorial libraries of antibody molecules which can be produced from a variety of sources, and include naive libraries, modified libraries, and libraries produced directly from human donors exhibiting a specific immune response. In addition to combinatorial libraries, standard methods for producing antibodies can be utilized to provide templates for domain exchanged binding molecules of the invention. Once generated, mutagenesis techniques, as known to those of skill in the art, can be utilized to screen for mutations that provide high affinity binding to antigens by crystal structure determination or sequence determination, for example, as well as binding studies with antigens of interest.

In particular, it is desirable that the domain exchanged binding molecules of the invention have three important characteristics. First, it is important that there are amino acid residues present for stabilizing and favoring the V_{H}-V_{H} interface. By way of example, in the 2G12 antibody illustrated herein, the proline at residue 113 of the Heavy chain appears to be important for promoting the V_{H}-V_{H} domain swapping while valine at position 84 of the Heavy chain appears to be important for stabilization of the resulting V_{H}-V_{H} interface. In addition, in 2G12, it appears that isoleucine at position 19 of the Heavy chain, arginine at position 57 of the Heavy chain and phenylalanine at position 77 of the Heavy chain are also involved in stabilization of the V_{H}-V_{H} interface.

Second, the linker region between the heavy chain variable region (V_{H}) and the heavy chain constant region (C_{H}) from the standard ball and socket joint to extend into an adjacent Fab, for example, provides for domain exchange and allows a “kinking” of the molecule below the V_{H}-V_{H} interface.

Third, the interaction between the V_{H} and V_{L} domains are typically conserved in conventional antibodies to promote stabilization. In particular Gln^{L38} and Gln^{H39} are typically conserved (94% and 97% respectively). When these residues are altered or absent, they weaken the V_{H} and V_{L} interface, which is desirable for the domain exchanged binding molecules of the invention.

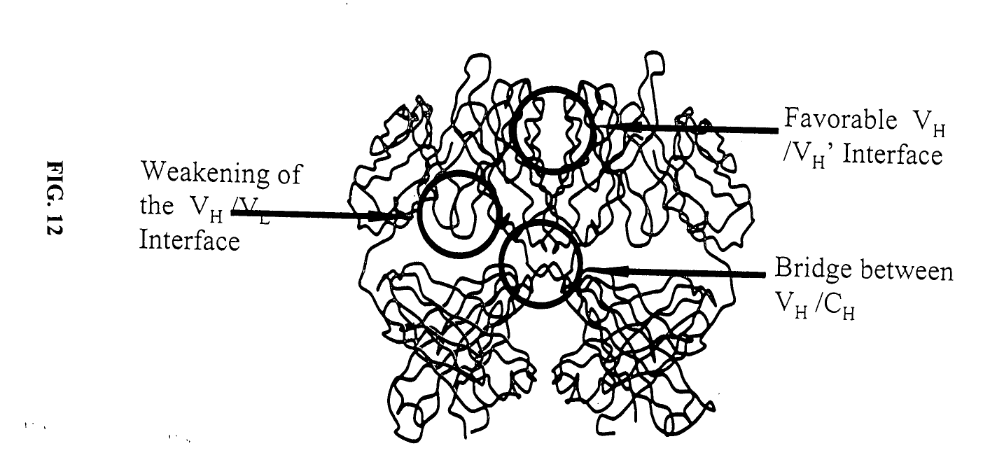

Given the teachings herein and the illustrative example provided by 2G12, one of skill in the art could generate other domain exchanged binding molecules as described in the present invention, especially having the three characteristics described above and shown in FIG. 12. It should be understood that while all three characteristics are desirable, it is possible that less than three will suffice to produce a domain-exchanged binding molecule of the invention.

The combinatorial library production and manipulation methods have been extensively described in the literature, and will not be reviewed in detail herein, except for those feature required to make and use unique embodiments of the present invention. However, the methods generally involve the use of a filamentous phage (phagemid) surface expression vector system for cloning and expressing antibody species of the library. Various phagemid cloning systems to produce combinatorial libraries have been described by others. See, for example the preparation of combinatorial antibody libraries on phagemids as described by Kang et al., Proc. Natl. Acad. Sci., USA, 88:4363-4366 (1991); Barbas et al., Proc. Natl. Acad. Sci., USA, 88:7978-7982 (1991); Zebedee et al., Proc. Natl. Acad. Sci., USA, 89:3175-3179 (1992); Kang et al., Proc. Natl. Acad. Sci., USA, 88:11120-11123 (1991); Barbas et al., Proc. Natl. Acad. Sci., USA, 89:4457-4461 (1992); and Gram et al., Proc. Natl. Acad. Sci., USA, 89:3576-3580 (1992), which references are hereby incorporated by reference.

The method for producing a conventional human monoclonal antibody generally involves (1) preparing separate H and L chain-encoding gene libraries in cloning vectors using human immunoglobulin genes as a source for the libraries, (2) combining the H and L chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular H and L chain-encoding genes and antibody molecules that immunoreact with the preselected antigen.

For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in the complementarity determining region (CDR) of the variable region of the immunoglobulin polypeptide, and subsequently screened for desirable immunoreaction and neutralization capabilities.

Similarly, the domain exchanged binding molecules of the invention can be generated by combinatorial library techniques wherein the V_{H}-V_{H} interface provides a framework for the molecules and the antibody combining sites (e.g, HCDR3) are randomized to produce a plurality of domain exchanged binding molecules with various antigen specificity and affinity. It is optional whether one or more loops of the CDR are randomized in a library. The libraries are typically expressed in phage, however, yeast, ribosome display or other systems known to those of skill in the art are also useful in the methods of the invention. The library is screened with an antigen of interest, for example, an array of gangliosides or other repeating units or a tumor cell. Novel domain exchanged binding molecules are selected as binding to an antigen of interest after panning the library.

As a further characterization of the binding molecules of the present invention, the nucleotide and corresponding amino acid residue sequence of the molecule's H or L chain encoding gene is determined by nucleic acid sequencing. The primary amino acid residue sequence information provides essential information regarding the binding molecule's epitope reactivity.

As used herein, the term “vector” refers to a nucleic acid molecule capable of transporting between different genetic environments another nucleic acid to which it has been operatively linked. Preferred vectors are those capable of autonomous replication and expression of structural gene products present in the DNA segments to which they are operatively linked. Vectors, therefore, preferably contain the replicons and selectable markers described earlier.

As used herein with regard to DNA sequences or segments, the phrase “operatively linked” means the sequences or segments have been covalently joined, preferably by conventional phosphodiester bonds, into one strand of DNA, whether in single or double stranded form. The choice of vector to which transcription unit or a cassette of this invention is operatively linked depends directly, as is well known in the art, on the functional properties desired, e.g., vector replication and protein expression, and the host cell to be transformed, these being limitations inherent in the art of constructing recombinant DNA molecules.

The level of expression of cell surface carbohydrate antigens is often significantly increased on carcinogenic transformation, and, in some cases, the expression of particular antigens appears to be associated primarily with the transformed state. Thus, carbohydrate-based antigens offer the potential for a targeted immunotherapeutic approach to the treatment of certain forms of cancer and metastases. The development of effective cancer vaccines based on carbohydrate antigens is an extremely challenging undertaking, however, and there are potential impediments to the success of such an endeavor. The first of these is related to the inherently low immunogenicity that the native carbohydrate antigens may exhibit. To mount an effective active immune response, this immune tolerance to the “self-antigens” must be overcome. Another factor that must be addressed en route to the development of carbohydrate-based cancer vaccines is that their isolation from natural sources is an extremely arduous task, and typically results in only minute quantities of material being obtained. Although the realization of an immunological approach to cancer control using carbohydrate-based vaccine constructs is clearly a nontrivial undertaking, efforts of this sort appear well justified, as there is considerable evidence supporting the notion that naturally acquired, actively induced, or passively administered antibodies directed against carbohydrate antigens are able to mitigate against circulating tumor cells and micrometastases. Thus, in another embodiment, the invention provides a method of treating cancer and metastases in a subject, including administering to the subject an antibody designed by a method of the invention. Such antibodies show higher affinity for carbohydrate antigens and repeating motifs, for example.

The following examples are intended to illustrate but not limit the invention.

### EXAMPLE 1

### Materials and Methods

Preparation of Man₉GlcNAc₂ oligosaccharide was performed by hydrazinolysis. Soy bean agglutinin was purchased from Sigma (L-1395, Lectin from Glycine Max). One hundred mg of the glycoprotein was dissolved in 0.1% trifluoroacetic acid, dialyzed against it, lyophilized and cryogenically dried for 48 h. The sample was dissolved in 5 ml of anhydrous hydrazine under argon and heated at a rate of 10° C./h and held at 85° C. for 12 h. Excess hydrazine was removed by evaporation under vacuum, followed by addition and evaporation of 5 ml toluene five times. After dissolving the released glycan with 9 ml of a saturated solution of sodium bicarbonate on ice, re-N-acetylation was performed by addition of 1.1 ml of acetic anhydride with gentle agitation, a further 1.1 ml of acetic anhydride added after 10 min, and the mixture incubated at room temperature for 50 min. The acetylated sample was filtered through No.54 filter paper (Whatman), and sodium salts in the filtrate were removed with Dowex AG50W-X12 (H⁺) column (2 cm×15 cm, 200-400 mesh, Bio-Rad), and the glycan was eluted with 300 ml of water five times. All the eluate was lyophilized and dissolved in 15 ml water, 15 ml ethanol and 60 ml n-butanol sequentially. Peptides were removed by cellulose column (1.5×25 cm) equilibrated with butanol:ethanol:water (4:1:1, v/v/v), washed with 6 column volumes of the solution, followed by 1 column volume of absolute ethanol. The glycan was eluted with water in 3 ml×20 fractions. Carbohydrate positive fractions were identified by phenol-sulfuric acid method (74). As reported by Lis and Sharon (23), the glycan structure was confirmed to be Man₉ by HPLC after fluorescent labeling with 2-aminobenzamide (24, 25) and MALDI-TOF mass spectrometry.

### EXAMPLE 2

### Crystal Structure Determination

Crystal structure determination was performed as described below. Human monoclonal antibody 2G12 (IgG1,κ) was produced by recombinant expression in Chinese hamster ovary cells. Fab fragments were produced by digestion of the immunoglobulin with papain followed by purification on protein A and protein G columns, and then concentrated to ˜30 mg/mL. Unliganded Fab 2G12 crystals were grown by the sitting drop vapor diffusion method with a well solution (1 mL) of 1.05M ammonium sulfate, 18% PEG 6000, and 0.1M imidazole malate, pH 6.0. Fab 2G12 was mixed with Manα1-2Man at a 5:1 (carbohydrate:Fab) molar ratio. Fab 2G12+Manα1-2Man crystals were grown from 2M Na/K phosphate, pH 7.0. Man₉GlcNAc₂ was also mixed with Fab 2G12 at a 5:1 (carbohydrate:Fab) molar ratio, and crystals grown from a well solution of 25% PEG 400, 0.2M imidazole malate, pH 7.0. In all cases, 1 μl of protein was mixed with an equal volume of reservoir solution. For all crystals, data were collected at the Stanford Synchotron Radiation Laboratory (SSRL) beamline 11-1 at 100K. Unliganded Fab 2G12 crystals were cryoprotected by a quick plunge into a reservoir solution containing 20% ethylene glycol, while Fab 2G12+Manα1-2Man were cryoprotected similarly with 25% glycerol. Fab 2G12+Man₉GlcNAc₂ crystals required no cryoprotectant. Unliganded Fab 2G12 data and the Manα1-2Man complex were reduced in orthorhombic space group P2₁2₁2 with unit cell dimensions a=76.8 Å, b=94.2 Å, c=171.1 Å and a=81.7 Å, b=94.0 Å, c=169.2 Å respectively. Fab 2G12+Man₉GlcNAc₂ data were reduced in orthorhombic space group I222 with unit cell dimensions a=135.8 Å, b=145.7 Å, c=148.6 Å. All data were indexed, integrated, and scaled with HKL2000 (26) using all observations >−3.0σ.

The Matthews coefficient (Vm) (27) for the unliganded Fab 2G12 was estimated as 3.16 Å³/dalton, with two Fab molecules per asymmetric unit. For unliganded Fab 2G12, rotation functions were performed with AMoRe (28) against our library of 125 intact Fab molecules separated into individual variable and constant domains. The strongest rotation and translational solutions were found from the variable and constant domains of Fab 1fvd (29). Positional refinement of the four individual domains (the variable and constant regions from each of the Fab molecules in the asymmetric unit) gave an overall correlation coefficient of 57.2% and an R-value of 40.8%. The Fab 1fvd model was then “mutated” to the correct sequence and rebuilt using TOM/FRODO (30), and refined with CNS version 1.1 (31) and REFMAC using TLS refinement (28). Refinement and model building were carried out using all measured data (with F>0.0σ). Tight non-crystallographic symmetry restraints were applied early on the model building and released gradually. Electron density maps for model building included 2Fo-Fc, Fo-Fc, and composite annealed omit 2Fo-Fc maps. An R_{free} test set consisting of 5% of the reflections was maintained throughout refinement.

The final refined structure for the unliganded Fab 2G12 was then used as a molecular replacement solution for Fab 2G12+Manα1-2Man. Molecular replacement with AMoRe gave a correlation coefficient of 64.2% and an R value of 35.8%. The structure was then built and refined in a similar manner to the unliganded Fab 2G12. Although the data from 1.75 Å to 1.6 Å have an acceptable I/σ (˜2.0), they were fairly incomplete (˜40%), and were included during model building but omitted from the final statistics.

The 1.75 Å structure of Fab 2G12 Manα1-2Man was used as the molecular replacement probe in AMoRe for Fab 2G12+Man₉GlcNAc₂ (correlation coefficient of 61.8% and R value of 42.2%). Man₉GlcNAc₂ was initially built using a model of Man₉GlcNAc₂ with ideal torsion angles and frequently seen rotamers (32), which was then adjusted to fit the electron density.

Sc coefficients (33) and buried molecular surface calculations were performed using the programs SC (34) and MS (35), in which a 1.7 Å probe radius and standard van der Waals radii were used (36). The Sc coefficients here represent a tightly packed interface typical of those found in oligomeric protein structures (which have Sc coefficients that range from 0.70 to 0.76 (33)). As this V_{H}/V_{H}′ interface is found in all three independent crystal structures of Fab 2G 12, all measurements and analysis described here will use the highest resolution structure (1.75 Å) of Fab 2G12 complexed with Manα1-2Man.

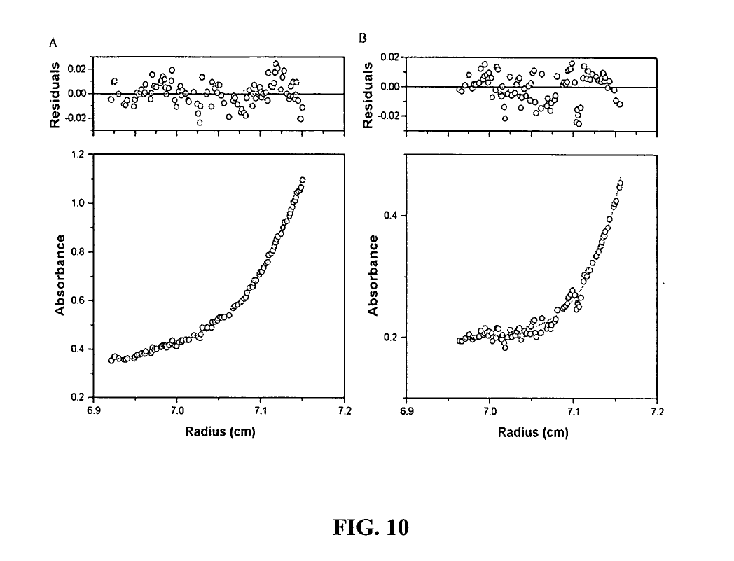

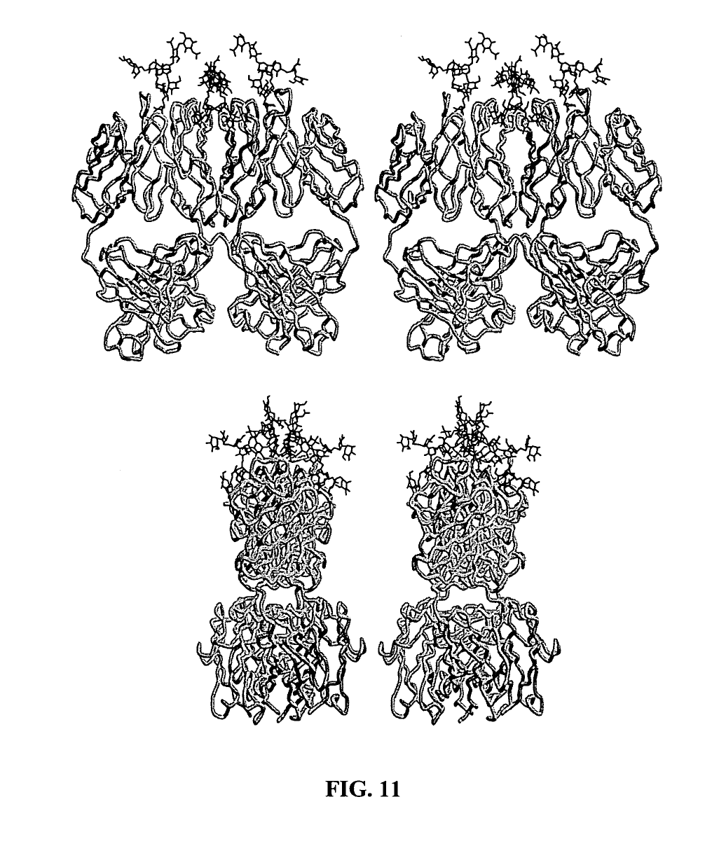

The hydrodynamic molecular weights of the Fab 2G12 and a control Fab (Fab NC-1) were determined by sedimentation equilibrium measurements employing a temperature-controlled Beckman XL-I Analytical Ultracentrifuge equipped with an An-60 Ti rotor and a photoelectric scanner (Beckman Instrument Inc., Palo Alto, Calif.). Protein samples were loaded in a double sector cell equipped with a 12 mm Epon centerpiece and a sapphire optical window. The reference compartment was loaded with the matching phosphate buffered saline (PBS) solution (100 μL). Samples (100 μg protein in 80 μL PBS buffer) were monitored employing a rotor speed of 3000 to 20000 rpm at 25° C. and analyzed by a nonlinear squares approach using Origin software (Microcal Software Inc., Northampton, Mass.) using appropriate models (i.e. single species model and two species models (37, 38)). The sedimentation equilibrium profiles of Fab 2G12 and Fab NC-1 were substantially different, with Fab NC-1 fitting to the expected single species model with apparent molecular weight corresponding to Fab monomer, while the Fab 2G12 data fitted to a two-species model with molecular weight corresponding to a mixture of monomer and dimer. Based on the absorbance, the mole ratio between monomer and dimer is 1:2, corresponding to 80% of the Fab 2G12 molecules existing as part of a dimer in solution (FIG. 10). For gel filtration of Fab 2G12, 100 μg of Fab 2G12 was loaded in PBS (200 μl) onto a Superdex 200 HR 10/30 column (Pharmacia). The column was equilibrated in PBS (2× column volume) and then protein was eluted in PBS (flow rate 0.5 mL/minute). The protein was detected by UV absorbance.

Sedimentation velocity of 2G12 IgG was used to determine the sedimentation coefficient of 2G12 IgG1 and relate it to that of other IgG1's (2F5, b6, and b12). Proteins (50 μg each) were dialyzed in PBS buffer. The data were collected on a temperature-controlled Beckman XL-I analytical ultracentrifuge (equipped with a An60Ti rotor and photoelectric scanner). A double sector cell, equipped with a 12 mm Epon centerpiece and sapphire windows, was loaded with 400-420 μL of sample using syringe. Data were collected at rotor speeds of 3000-50000 rpm in continuous mode at 25° C., with a step size of 0.005 cm employing an average of 1 scan per point and analyzed using the program Sedfit (39).

Competition enzyme-linked immunosorbent assays (ELISAs) were performed to determine the relative inhibition constants of different carbohydrates on 2G12. GP120_{JR-FL} was first captured onto microtiter plate wells (flat bottom, Costar type 3690; Corning Inc.). Subsequently serial dilutions of Man₉GlcNAc₂, Manα1-2Man, mannose, and other mono- and disaccharides were added to the wells in the presence of 2G12 (1 μg/ml). Subsequent blocking, washing, and detection steps were performed as described in (13).

Fab 2G12 Mutagenesis and Binding Assays: Point mutations were generated using the Quikchange mutagenesis kit™ (Stratagene). All mutations generated were verified by DNA sequencing. Single colonies were selected and placed onto SB media and carbenicillin. After six hours at 37° C., the cultures were placed at 30° C. and expression was induced overnight using 1 mM IPTG. Cells were centrifuged (5000×g for 5 minutes) and the protein was extracted from the pellet through 5 freeze-thaw fracture cycles. ELISAs were performed on the crude Fab supernatants to determine the relative binding affinity of wild type and mutant Fab 2G12 to gp¹²⁰_{JR-FL}. One set of microtiter well plates were coated with gp120_{JR-FL} to capture Fab 2G12. Blocking, washing, and detection steps were performed as described in (13). To normalize for expression of Fab, unconjugated goat-anti-human-F(ab′)₂ antibody (1 μg/mL in PBS, Pierce) was used to capture mutant Fab 2G12 from supernatant. After blocking and washing, the plate was then probed with goat-anti-human-F(ab′)₂-AP (0.6 μg/mL, Pierce). Apparent affinities were calculated as the antibody concentration at 50% maximal binding; changes in affinity were expressed as {(apparent affinity of wild type Fab 2G12)/(apparent affinity of mutant Fab 2G12)}×100%.

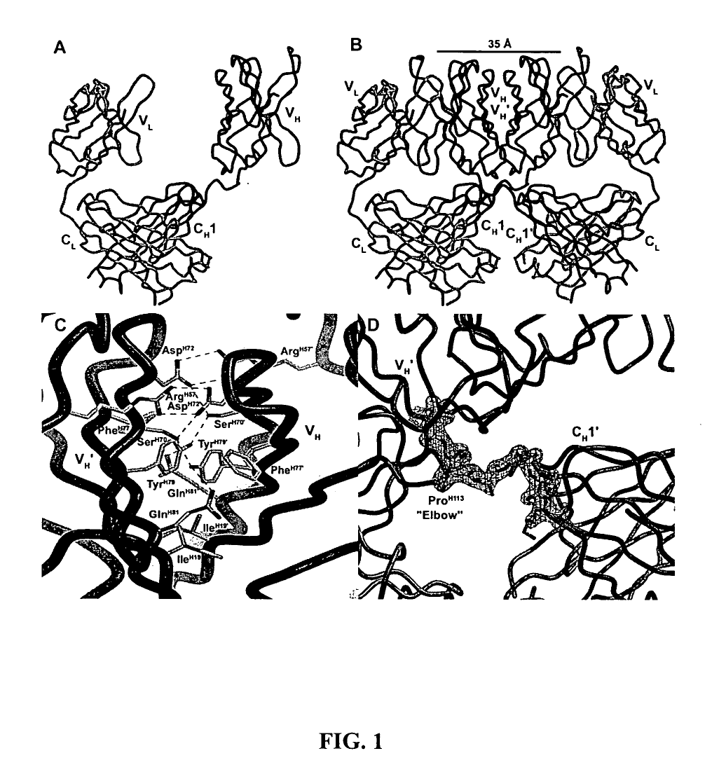

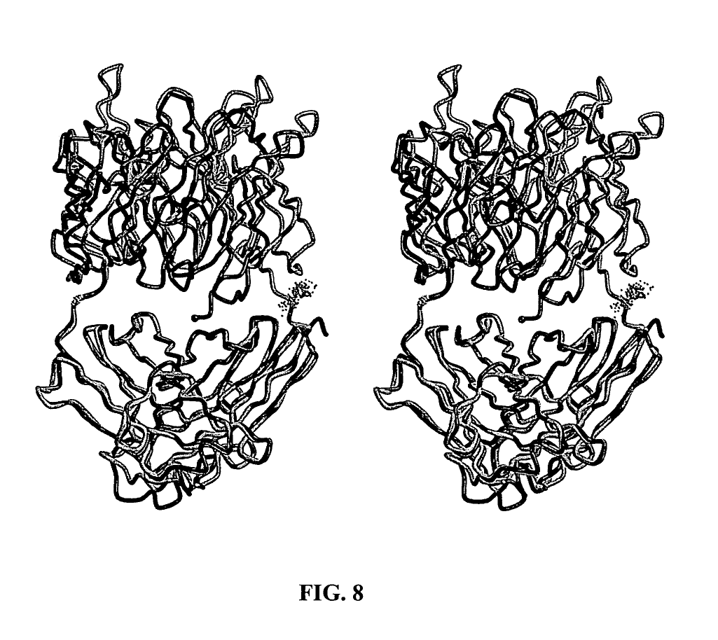

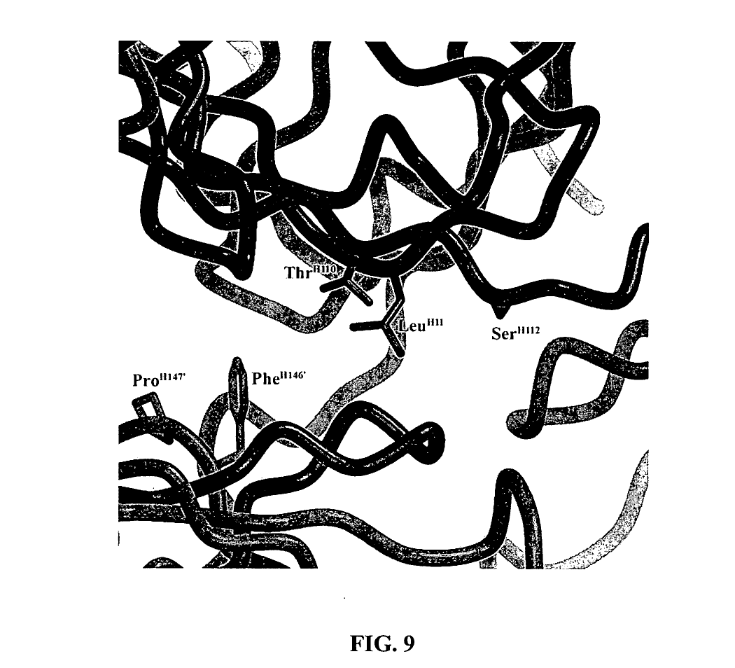

Crystal structures of Fab 2G12. Crystal structures were determined for the unliganded Fab 2G12 at 2.2 Å resolution, for the Fab bound to oligosaccharide Man₉GlcNAc₂ at 3.0 Å, and for the Fab bound to disaccharide Manα1-2Man at 1.75 Å (Table 1). The asymmetric unit in each crystal form contains two Fab molecules, which turns out in this case to be of major interest because of their unusual oligomeric arrangement. The two independent Fab molecules are intertwined via a three-dimensional swap (40) of their V_{H} domains (FIGS. 1A, B) to form a structure that has not been observed previously in over 250 Fab structures deposited in the Protein Data Bank. This exchange of the V_{H} domains creates a tightly-packed dimeric assembly of two Fabs. While the variable (V_{H}, V_{L}) and constant regions (C_{H}1, C_{L}) are each structurally similar to their corresponding domains in other Fab molecules, the variable regions in 2G12 are twisted with respect to the constant region from their normal architecture in a typical Fab so as to accommodate the V_{H} domain exchange (FIG. 8). This V_{H} domain-exchanged dimer lacks the highly conserved ball-and-socket joint (41) between V_{H} and C_{H}1 that is believed to play a key role in the flexibility of the variable domains with respect to the constant domains, although the conserved ball-and-socket residues are still present (FIG. 9).

The V_{H} domains within the dimer are related by a non-crystallographic two-fold symmetry axis of 178.5°, such that the two Fabs are arranged side-by-side with their respective combining sites facing in the same direction and separated by approximately 35 Å.

Analysis of the Fab 2G12 structure reveals three factors, mainly as a result of somatic mutation, that appear to promote domain exchange: weakening of the V_{H}/V_{L} interface (closed interface), an unusual sequence and structure of the elbow region connecting the V_{H} and C_{H}1 domains (hinge loop), and the creation of a favorable V_{H}/V_{H}′ interface (open interface). The “closed interface” refers to the interface between the swapped domain and the main domain that exists in both the monomer and the domain-swapped oligomer. The “hinge loop” is the segment of polypeptide chain that links the swapped domain and the main domain and adopts different conformations in the monomer and the domain-swapped oligomer. The “open interface” exists only in the domain-swapped oligomer, but not in the monomer. The interplay between these three factors (destabilization of the closed interface, conformational shift in the hinge loop, and an energetically favorable open interface) can promote domain swapping (40). Thus, all of the key factors previously shown to promote domain exchange and favor stabilization of the dimeric assembly over monomers are found here (40).

The V_{H}/V_{L} interface in 2G12 is perturbed by the absence of the highly conserved interaction between the V_{H} and V_{L} domains that is also conserved in αβ TCRs (42). Gln^{L38} and Gln^{H39} (94% and 97% conserved) usually hydrogen bond to each other at the base of the combining site, but in 2G12, position H39 is a rarely observed arginine residue (0.7%) that is too distant (almost 4 Å) from Gln^{L38} to interact. All measurements of residue occurrence are made using the Kabat sequence database (43).

Comparison of Fab 2G12 with other Fab structures shows that the connection between V_{H} and C_{H}1 is unusual, such that the V_{H} domain pivots around residue Pro^{H113} to promote domain swapping (FIG. 1D). A proline residue in the elbow region (or hinge loop) at H113 is relatively uncommon, occurring in only 1.8% of known sequences, with serine being by far the most prevalent residue (95.2%). This Fab structure represents the first described with proline at this position. Proline residues have been found frequently in connecting hinge loops in many other domain-swapped or oligomerizing proteins (reviewed in (40, 44)), and the unique phi constraints that a proline residue imposes on the peptide backbone appear to facilitate domain interchange. The new conformation of the hinge loop appears to be stabilized in the domain-exchanged structure by hydrophobic interactions between Pro^{H113} and Val^{H84}, which also is not commonly found at this position (Alanine is the most common residue at this position (58%)). Val^{H84} occurs in only 5% of known antibody sequences and is sometimes found at the same time as Pro^{H113} in other antibodies (31).

The newly formed V_{H}/V_{H}′ interface is remarkably complementary (S_{c} coefficient 0.73), as illustrated by an extensive hydrogen bonding and salt bridge network (FIG. 1C)(33) with a total of 10 hydrogen bonds, as well as 136 van der Waals interactions (46). Of the hydrophilic interface residues, only Arg^{H57} is uncommon (1.4%). In addition, π-stacking interactions occur between residues Tyr^{H79} and Tyr^{H79′}. Residues marked with an ′ are to indicate they correspond to the second Fab molecule of the domain-exchanged Fab dimer. At the bottom of this interface, an extensive hydrophobic patch is created from the confluence of Ile^{H19}, Ile^{H19′}, Phe^{H77}, Phe^{H77′}, Tyr^{H79′} and Tyr^{H79}. Ile^{H19} and Phe^{H77} are rare occurrences at these positions in V_{H} sequences (0.11% and 0.19% respectively) and arise from somatic mutation. A total of 1,245 Å² of molecular surface is buried at this V_{H}/V_{H}′ interface, which is significant compared to the standard V_{H}/V_{L} interface in antibodies which here buries 1,690 Å² of molecular surface.

### EXAMPLE 3

### Oligomeric State of 2G12 in Solution

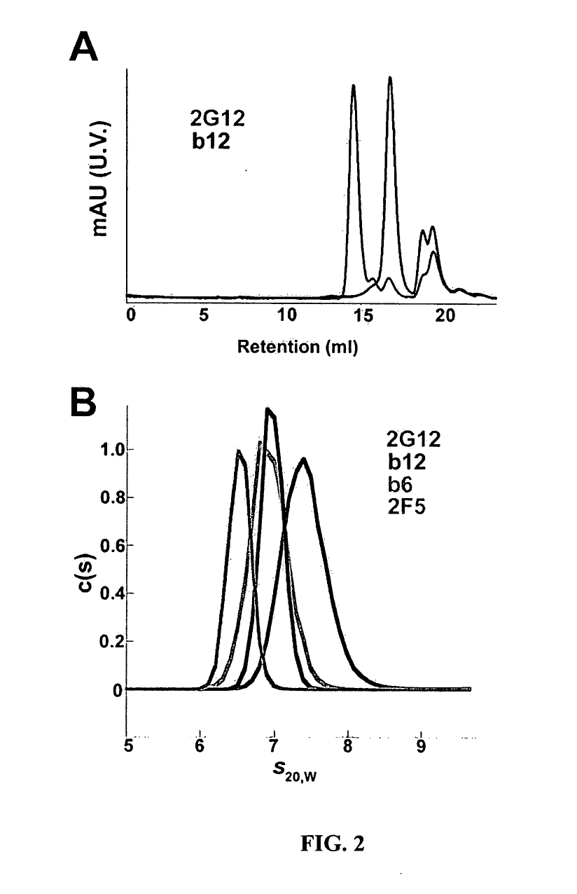

These crystallographic observations prompted us to investigate whether or not the 2G12 Fab dimer exists in solution or is simply an artifact of crystallization. We examined the Fab oligomeric state by sedimentation equilibrium analytical ultracentrifugation and by gel filtration (FIG. 2A)(see above).

In gel filtration experiments (see FIG. 2A-B), Fab 2G12 elutes from the column at a molecular weight of ˜100 kDa, while a control Fab (b12) elutes at ˜50 kDa. The molecular weights suggest that Fab 2G12 exists almost entirely as a dimer in solution, whereas Fab b12 is present as the expected monomer. The completeness of the papain digests and the molecular weights of the Fab monomers were confirmed by SDS-PAGE (data not shown). Furthermore, the s_{20,W} value of 2G12 IgG1 was significantly higher (7.39) than other IgG1 molecules, which had s_{20,W} values between 6.50 and 6.89. Previously published s_{20, W} values for IgG1 molecules are around 6.6 (70, 71). Thus, 2G12 is an outlier, in agreement with the elongated structure of the IgG1 that would arise from the domain-swapped dimer of its Fabs.

In both experiments, Fab 2G12 exists predominantly (80-100%) as a dimer in solution. We next examined the conformation of the intact IgG1 2G12, in order to rule out the possibility that the Fab is only capable of domain swapping when untethered from the Fc fragment of the IgG. Previous studies have shown that truncation of some proteins can lead to artificial domain swaps which do not or can not occur in the native, intact protein, for example Domain 5 of TrkA, TrkB, and TrkC (47). Also, domain swaps in engineered Fv fragments have been identified through variation of the length of the linker region between V_{H} and V_{L}, as for example in diabodies (48) and triabodies (49) in which the natural V_{H}/V_{L} pairing is perturbed due to the shortness of the linker connection. The sedimentation coefficient (s_{20, W}) of 2G12 is unusually high when compared to other IgG1's and previously published values (FIG. 2B), consistent with a more compact linear configuration, as opposed to a Y- or T-shape of the typical antibody molecule. Furthermore, a recent negative stain electron microscopy study of 2G12 bound to SOS gp140 (a covalently-constrained gp120/gp41 molecule) provided clear images of the antibody in an unusual, extended linear conformation (50), as compared to the normal Y- or T-shaped configuration seen for other anti-HIV-1 antibodies. Therefore, these data are all consistent with domain-swapping of the Fabs in 2G12 whether as Fab fragments, the intact IgG1, or when the IgG1 is complexed to gp120.

### EXAMPLE 4

### Carbohydrate Specificity and Binding Sites of 2G12

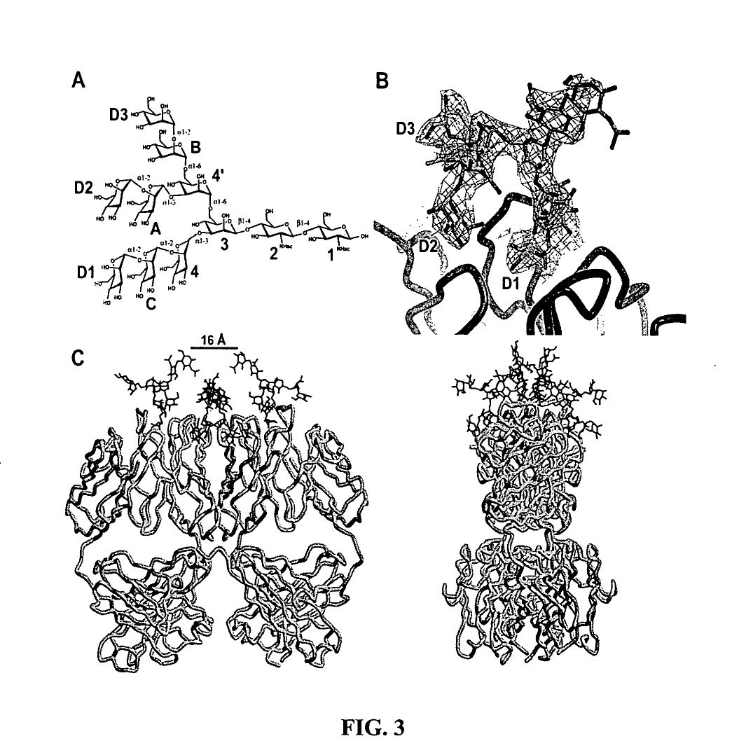

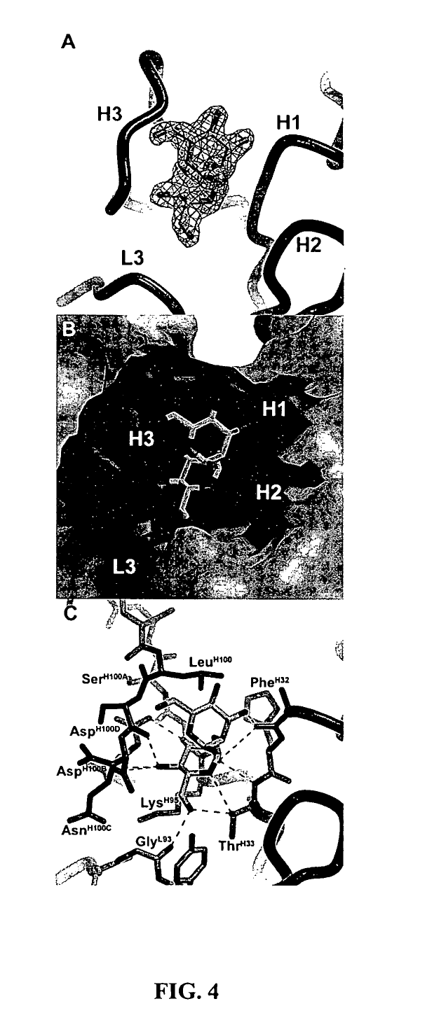

Previous data had indicated that 2G12 recognizes Man₉GlcNAc₂ moieties (FIG. 3A) covalently attached to gp120 (13, 14). To explore the binding specificity, we co-crystallized Fab 2G12 with Man₉GlcNAc₂. Although the co-crystals were highly anisotropic and diffracted only to modest resolution (3 Å), the electron density for the Man₉GlcNAc₂ is unusually well defined for a carbohydrate ligand (FIG. 3B), albeit with an increase in B values (as expected) farther from the protein surface. The two branching points (at sugars 3 and 4′, FIG. 3A) of the Man₉GlcNAc₂ are clearly visible and led to an unambiguous interpretation of the electron density. Our previous studies showed that the disaccharide Manα1-2Man could also bind to 2G12 (13). Hence, we co-crystallized 2G12 with this disaccharide. In this high resolution structure (1.75 Å) of the complex, the Manα1-2Man density is extremely well defined and its conformation is within the preferred range for this particular disaccharide (FIG. 4A)(51). A surprising conclusion from these two independent 2G12 crystal structures with Man₉GlcNAc₂ and Manα1-2Man is that the 2G12 domain-exchanged dimer contains multiple, distinct binding sites for carbohydrate: two correspond to the normal antibody combining site and two to novel sites within the V_{H}/V_{H}′ interface generated in the domain exchange (FIG. 3C).

Primary combining site. In the Man₉GlcNAc₂ complex, 2G12 contacts four sugars (3, 4, C, and D1) in the D1 arm with the majority of contacts (85%) being with the terminal Manα1-2Man disaccharide (FIGS. 3A, 3B, 3C). In the disaccharide complex, Manα1-2Man occupies only the two conventional combining site pockets, which are separated by about 35 Å, and suggests that this represents the higher affinity site for this particular mannose linkage. The 2G12 contact residues with the disaccharide in the antigen binding pocket (FIG. 4B) are L93-94 (CDR L3), H31-33 (CDR H1), H52a (CDR H2), and H95-H100D (CDR H3). A total of 226 Å² of molecular surface from Fab 2G12 and 220 Å² of molecular surface from Manα1-2Man is buried during complex formation, with a total of 12 hydrogen bonds and 48 van der Waals interactions in each antigen binding site (FIG. 4C).

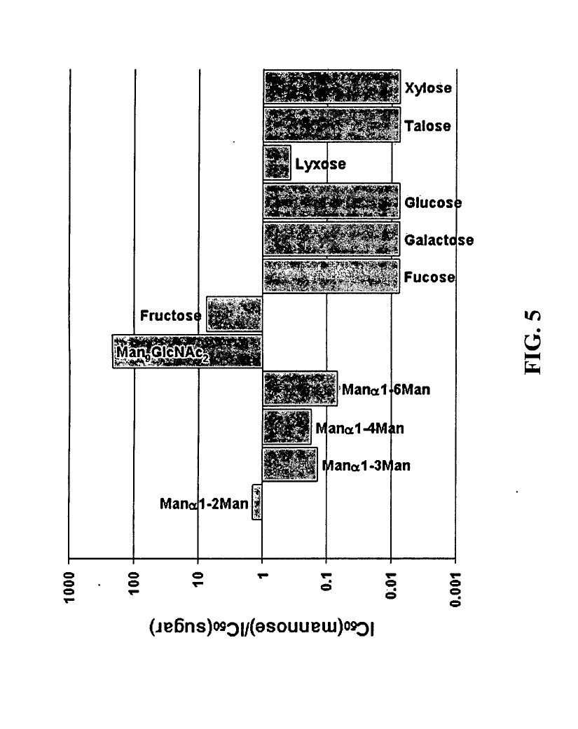

Competition studies confirm that the Manα1-2Man interaction alone cannot account for the large increase in affinity observed when 2G12 binds to Man₉GlcNAc₂ (FIG. 5). As illustrated in FIG. 5, Man₉GlcNAc₂ inhibits binding of Mab 2G12 to gp120_{JR-FL} by over 200-fold compared to mannose and by over 50-fold compared to the disaccharide Manα1-2Man. Fructose is a better inhibitor than mannose. The structure of fructose, when docked into the primary combining site, can mimic positions of four of the oxygen atoms of mannose, and can also potentially make further hydrogen bonding interactions compared to mannose. No other simple sugars or mannose disaccharides with other linkages inhibit 2G12 binding to gp120.

The additional antibody contacts with sugars 3 and 4 in the primary combining site presumably provide extra favorable interactions with Man₉GlcNAc₂, as compared to Manα1-2Man. Asp^{H100B}, which is oriented differently in the Man₉GlcNAc₂ and Manα1-2Man complexes, hydrogen bonds to the branching sugar 3 of the Man₉GlcNAc₂, while Tyr^{L94} hydrogen bonds to mannose 4. In the 2G12-Man₉GlcNAc₂ complex, the buried surface area is larger, ranging from 350-450 Å² of molecular surface for the Fab and 330-450 Å² from Man₉GlcNAc₂ in the two antigen binding sites.

The specificity of the primary combining site of 2G12 for Manα1-2Man at the tip of D1 arm of Man₉GlcNAc₂ results from a combination of several structural factors. First, the primary combining site forms a deep pocket that can only accommodate terminal sugar residues. Second, 2G12 can selectively bind Manα1-2Man in the primary combining site due to the highly complementary geometry of the hydrogen bonds between 2G12 and the sugar residues. Lastly, the specificity is finely tuned for the interaction with the Manα1-2Man moieties at the tip of the D1 arm of Man₉GlcNAc₂ due to the additional specific interactions with the mannose 3 and mannose 4 sugars.

Secondary binding site. The V_{H}/V_{H}′ three-dimensional domain swap of Fab 2G12 creates a completely novel binding surface not seen before in any other antibody structure. The D2 arms of the symmetry-related Man₉GlcNAc₂ residues in the crystal interact with this composite surface of the V_{H}/V_{H}′ interface, providing for two additional binding sites (FIG. 3C). The V_{H}/V_{H}′ interface interactions are mainly with the central mannose A of the D2 arm, but contacts are also made with the D2 and 4′ sugars. Furthermore, the carbohydrate chain lies parallel to the surface in a shallow binding site and is not bound end-on in a deep pocket as in the primary combining site. Hence, it is not clear whether the secondary binding site is as specific for the D2 arm compared to the highly specific D1 arm interaction in the primary binding site. In this structure, the corresponding D1 arm of the same Man₉GlcNAc₂ is found in the higher affinity primary combining site of a crystallographically-related Fab 2G12 molecule. Thus, it is possible that the secondary binding site could also interact with D1 or D3 arms, but these interactions are not observed here due to crystal packing.

The two independent Man₉GlcNAc₂ moieties in the asymmetric unit differ slightly in their interaction with the V_{H}/V_{H}′ interface, but a total of 280-310 Å² of molecular surface from Fab 2G12 and 250-290 Å² of molecular surface from Man₉GlcNAc₂ is buried during complex formation. Eight to nine hydrogen bonds and 22-26 van der Waals contacts are made in each V_{H}/V_{H}′ interface binding site. While these secondary binding site interactions are formed from the juxtaposition of four Fab-carbohydrate complexes in the crystal lattice, these additional binding sites arise from the unique assembly of the domain-exchanged Fabs and likely emulate the high affinity interaction of the antibody with the dense array of oligomannose sugars on the surface of gp120.

### EXAMPLE 5

### Mutagenesis of 2G12

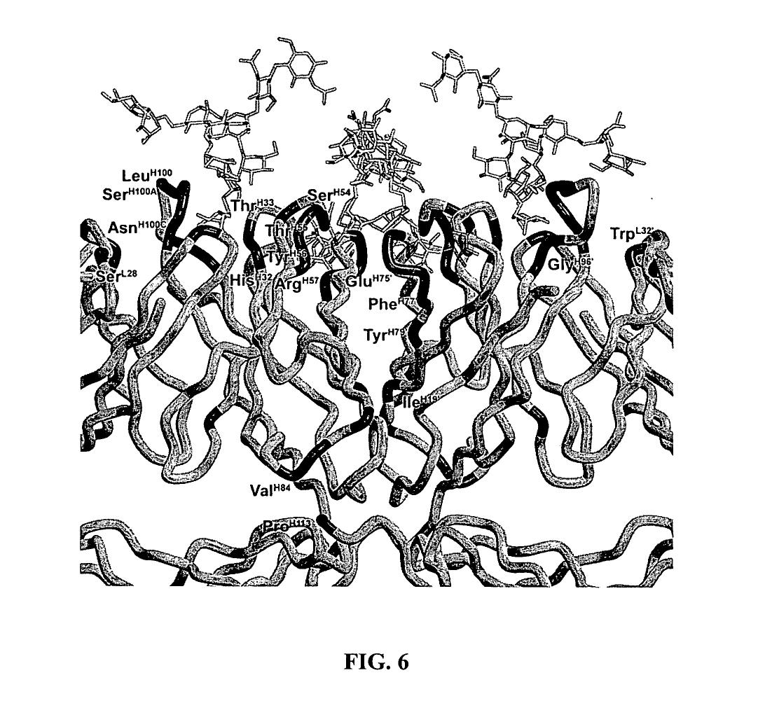

Mutagenesis of Fab 2G12 was carried out to investigate the role of domain exchange and multivalent interactions in the binding of 2G12 to gp120. Residues in 2G12 that were suspected to play a role in domain exchange, as well as in ligand binding, were substituted by alanine residues and assayed for binding to gp120_{JR-FL} (FIG. 6; Table 2). In some instances where the germline residues or somatic mutations involved were rare, reverse mutations to the residue encoded by the closest germline gene were introduced. Alanine substitution of many of the residues that make up the primary combining site abolished 2G12 binding to gp120_{JR-FL}. More notable, however, were the effects of alanine substitutions on residues located in the V_{H}/V_{H}′ interface. Almost all of these substitutions resulted in decreased binding to gp120_{JR-FL}. Importantly, alanine or serine substitution of Pro^{H113} completely abrogated binding of 2G12 to gp120_{JR-FL}. Alanine substitution of Val^{H84}, which interacts with Pro^{H113} also led to a substantial decrease in binding. In addition, alanine substitutions of many of the residues involved in binding the D2 arm of Man₉GlcNAc₂ in the secondary binding site decreased gp120 binding and provided further evidence that the unique V_{H}/V_{H}′ interface plays a role in multivalent binding of 2G12 to gp120.

### EXAMPLE 6

### Biological Significance of the 2G12 Domain-Swapped Dimer

We have presented compelling biochemical, biophysical and crystallographic evidence to illustrate that the V_{H} domains of antibody 2G12 exchange between its two adjacent Fab fragments so as to form an extensive multivalent binding surface composed of the two conventional combining sites and a novel homodimeric V_{H}/V_{H}′ interface. The 2G12 V_{H}/V_{H}′ interface is composed from many conserved germline-encoded residues, but with three uncommon mutations (Ile^{H19}, Arg^{H57}, and Phe^{H77}) that appear to promote stabilization of this novel interaction. The proline at position H113 also appears to promote this V_{H}/V_{H}′ domain exchange and the unusual extended conformation of the hinge peptide in the elbow region appears to be stabilized by hydrophobic interactions between Pro^{H113} and Val^{H84}. Analysis of the Kabat antibody sequence databases yielded no other heavy chain sequences with the exact combination of Ile^{H19}, Arg^{H57}, Phe^{H77}, and Pro^{H113} (45), presumably because they arise from independent somatic mutation events. However, one could certainly envision that other combinations of mutations could promote domain exchange and favorable V_{H}/V_{H}′ interactions.

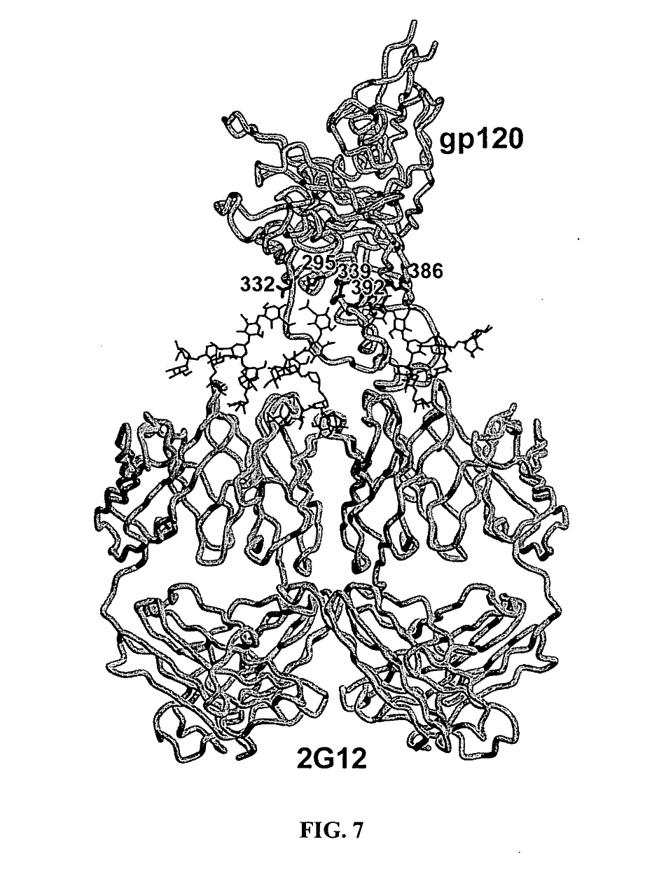

Recognition of carbohydrates on HIV-1 by an antibody poses a series of problems. The novel structure of 2G12 represents an elegant molecular solution. First of all, an antibody response to the carbohydrates on HIV-1 would appear to be excluded by tolerance mechanisms. However, the dense cluster of oligomannose residues found on the “silent” face of gp120 has not been described for any other mammalian glycoprotein (52) and, hence, appears capable of eliciting an antibody response that is dependant on the proximity and spacing of the individual oligomannose moieties. Second, recognition of the dense cluster of carbohydrates is problematic for a conventional Y-or T-shaped IgG molecule. Geometrical constraints suggest that a single antibody combining site can bind only to carbohydrate residues from one oligomannose chain. Recognition of two oligomannose chains can only be achieved by bivalent antibody binding. It is conceivable that an IgG molecule could bivalently recognize two oligomannose chains 35 Å apart at their tips, as suggested for gp120 below (FIG. 7), but this would require a near parallel orientation of the two Fab arms that would be energetically disfavored. In contrast, the 2G12 domain-exchanged structure is well suited for recognition of two oligomannose chains at a spacing of about 35 Å. In this intertwined arrangement, there is no entropic penalty to be paid for bivalent attachment to the Fab arms, as in a conventional antibody, and, indeed, previous studies have shown that 2G12 binds with low entropy to gp120 (53). In addition, the V_{H}/V_{H}′ interface provides a completely novel surface that could act as an additional antigen binding region with which further oligomannose chains can interact and facilitate productive binding of 2G12 to a dense cluster of carbohydrates. Finally, as mentioned earlier, protein-carbohydrate interactions are notoriously weak and anti-carbohydrate antibodies typically have relatively low affinities in the submicromolar range (21). The oligomeric structure of 2G12 can lead to higher affinity (nM) by providing a virtually continuous surface for multivalent recognition with interaction sites that match the geometrical spacing of the carbohydrate array on gp120.

Comparison with other lectins. The proposed mode of binding of 2G12 is reminiscent of one of the suggested mechanisms of multivalent recognition by animal lectins, such as serum mannose-binding protein (MBP), in which avidity can be optimized by matching the appropriate geometrical arrangement of the binding sites in the lectin oligomer with the spacing of carbohydrate epitopes on the pathogen. Furthermore, as for 2G12, the specificity of mannose-binding proteins is achieved through multivalent interactions, as opposed to recognition via a single high affinity site (reviewed in (20, 54)).

DC-SIGN (dendritic cell specific intracellular adhesion molecule-3 grabbing nonintegrin), a C-type lectin, also binds carbohydrates on the envelope of HIV and facilitates viral infection of CD4⁺ T Cells (55). DC-SIGN differs from 2G12 in that it binds to an internal core feature of high-mannose oligosaccharides, as opposed to the terminal mannoses (56). Interestingly, it may be that HIV-1 has evolved oligomannose clusters in part to enhance binding to DC-SIGN by increased avidity through interactions (57) and 2G12 exploits this through its own unique multivalent recognition.

2G12 can also be compared with cyanovirin, a cyanobacterial lectin that neutralizes HIV-1 by binding carbohydrate on the surface of gp120 (58-60). Crystal structures of cyanovirin have shown that it is also capable of binding Manα1-2Man at the end of the D1 arm of Man₉GlcNAc₂ (61). Coincidentally, cyanovirin also can exist as a domain-swapped dimer (62) with four binding sites that can interact with gp120 (63). However, previous studies on cyanovirin have proposed that high affinity binding is achieved by interaction with only one oligomannose rather than a constellation of oligomannose moieties, as for 2G12 (13, 64).

2G12 recognition of HIV-1. From the crystal structures of 2G12 in complex with Man₉GlcNAc₂ and the gp120 core structure, we can now approximate how 2G12 might bind to gp120. Gp120 coordinates represent the 2.2 Å crystal structure of core gp120 from the H×B2 strain of HIV-1 complexed to CD4 and Fab 17b (65). The modeled V3 loop is as described in (18). The V4 loop was modeled by Mark Wormald (unpublished data). The overall three-dimensional conformation of Man₉GlcNAc₂, when covalenty attached to a protein surface is usually highly conserved (51). Hence, we can superimpose the Man₉GlcNAc₂ residues in the 2G12 complex onto their corresponding positions on the core of gp 120. Previous mutagenesis studies have implicated N-linked glycans at positions 295, 332, and 392 in gp120 as being most critical for 2G12 binding (13). In the glycosylated model, the Fab 2G12 dimer most likely binds gp120 at the N-linked glycans at positions 332 and 392 (FIG. 7). The terminal N-acetyl glucosamine residues of the Man₉GlcNAc₂ moieties in the primary combining sites of the Fab 2G12 dimer are ˜16 Å apart, while asparagine residues on gp120 at position 332 and 392 are similarly spaced ˜15 Å apart (but can vary between 14-20 Å depending on their rotamers). The glycan at 295 also appears to be important from this model because it is in close proximity to the glycan at 332, and, thus, its absence could increase the flexibility and perturb the conformation of glycan 332. Interestingly, this model also places the N-linked glycan at position 339 proximal to the V_{H}/V_{H}′ interface of the 2G12 Fab dimer. Although this glycan is not as critical for binding 2G12 as glycans at 295, 332, or 392, it could interact with the secondary, perhaps lower affinity, binding site at the V_{H}/V_{H}′ interface. However, there could also be some promiscuity in the oligomannose chains involved in 2G12 binding. It is possible that 2G12 could bind several combinations of the oligomannose chains on the surface of gp120, as long as they assume an appropriate geometric spacing.

The structures of Fab 2G12 complexed with Man₉GlcNAc₂ and Manα1-2Man are also provocative templates for innovative HIV-1 vaccine design. For example, the design of multivalent carbohydrate-based immunogens as vaccines has been proposed for targeting cancer cells (66). Immunogens designed to mimic the unique cluster of oligomannose sugars binding to antibody 2G12 can now be tested for their ability to elicit a 2G12-like immune response. The V_{H} domain-swapped Fab dimer represents a completely unexpected quaternary assembly for an antibody and reveals yet another paradigm for the way in which the immune system can respond to invasion by microorganisms. The 2G12 structure further provides a scaffold for engineering high affinity antibodies to molecular clusters, not only carbohydrates as might be found on pathogens and tumor cells, but also other clusters that might be naturally occurring or synthetic.

References

1. R. M. Zinkernagel, et al., Adv. Immunol. 79, 1-53. (2001).
2. D. R. Burton, Nat. Rev. Immunol. 2, 706-13. (2002).
3. F. Ferrantelli, R. M. Ruprecht, Curr. Opin. Immunol. 14, 495-502. (2002).
4. A. J. Conley, et al., Proc. Natl. Acad. Sci. USA 91, 3348-52. (1994).
5. C. E. Parker, et al., J. Virol. 75, 10906-11. (2001).
6. M. B. Zwick, et al., J. Virol. 75, 10892-905. (2001).
7. G. Stiegler, et al., AIDS Res. Hum. Retroviruses 17, 1757-65. (2001).
8. D. R. Burton, et al., Science 266, 1024-7. (1994).
9. P. Roben, et al., J. Virol. 68, 4821-8. (1994).
10. A. Trkola, et al., J. Virol. 69, 6609-17. (1995).
11. A. Trkola, et al., J. Virol. 70, 1100-8. (1996).
12. E. O. Saphire, et al., Science 293, 1155-9. (2001).
13. C. N. Scanlan, et al., J. Virol. 76, 7306-21. (2002).
14. R. W. Sanders, et al., J. Virol. 76, 7293-305. (2002).
15. R. Wyatt, et al., J. Virol. 67, 4557-65. (1993).
16. R. Wyatt, et al., Nature 393, 705-11. (1998).
17. P. D. Kwong, et al., Nature 393, 648-59. (1998).
18. P. D. Kwong, R. Wyatt, Q. J. Sattentau, J. Sodroski, W. A. Hendrickson, J. Virol. 74, 1961-72. (2000).
19. P. M. Rudd, R. A. Dwek, Crit. Rev. Biochem. Mol. Biol. 32, 1-100 (1997).
20. W. I. Weis, Curr. Opin. Struct. Biol. 7, 624-30. (1997).
21. I. A. Wilson, R. L. Stanfield, Nat. Struct. Biol. 2, 433-6. (1995).
22. R. Thomas, et al., J. Biol. Chem. 277, 2059-64. (2002).
23. H. Lis, N. Sharon, J. Biol. Chem. 253, 3468-76. (1978).
24. G. R. Guile, et al., Eur. J. Biochem. 258, 623-56. (1998).
25. J. C. Bigge, et al., Anal. Biochem. 230, 229-38. (1995).
26. Z. Otwinowski, W. Minor, Methods Enzymol. 276A, 307-326 (1997).
27. B. W. Matthews, Methods Enzymol. 114, 176-87 (1985).
28. CCP4, Acta Cryst. D50, 760-763 (1994).
29. C. Eigenbrot, M. Randal, L. Presta, P. Carter, A. A. Kossiakoff, J. Mol. Biol. 229 969-95. (1993).
30. T. A. Jones, in Computational Chemistry (Clarendon Press, Oxford, 1982) pp. 303-317.
31. A. T. Brünger, et al., Acta Cryst. D54, 905-21. (1998).
32. A. J. Petrescu, S. M. Petrescu, R. A. Dwek, M. R. Wormald, Glycobiology 9, 343-52. (1999).
33. M. C. Lawrence, P. M. Colman, J. Mol. Biol. 234, 946-50. (1993).
34. CCP4, Acta Cryst. D50, 760-763 (1994).
35. M. L. Connelly, J. Mol. Graphics 11, 139-141 (1993).
36. B. R. Gelin, M. Karplus, Biochemistry 18, 1256-68 (1979).
37. J. S. Philo, Anal. Biochem. 279, 151-63. (2000).
38. W. F. Stafford, 3rd, Anal. Biochem. 203, 295-301. (1992).
39. P. Schuck, Biophys. J. 78, 1606-19. (2000).
40. Y. Liu, D. Eisenberg, Protein Science 11, 1285-99. (2002).
41. A. M. Lesk, C. Chothia, Nature 335, 188-90. (1988).
42. K. C. Garcia, et al., Science 274, 209-19. (1996).
43. E. A. Kabat, T. T. Wu, H. M. Perry, K. S. Gottesman, C. Foeller, Sequences of proteins of immunological interest (U.S. Department of Health and Human Services, ed. Fifth, 1991), vol. 1.
44. M. Bergdoll, M. H. Remy, C. Cagnon, J. M. Masson, P. Dumas, Structure 5, 391-401. (1997).
45. A. C. Martin, Proteins 25, 130-3. (1996).
46. S. Sheriff, W. A. Hendrickson, J. L. Smith, J. Mol. Biol. 197, 273-96. (1987).
47. M. H. Ultsch, et al., J. Mol. Biol. 290, 149-159. (1999).
48. O. Perisic, P. A. Webb, P. Holliger, G. Winter, R. L. Williams, Structure 2, 1217-1226. (1994).
49. X. Y. Pei, P. Holliger, A. G. Murzin, R. L. Williams, Proc. Natl. Acad. Sci. 94, 9637-9642. (1997).
50. N. Schulke, et al., J. Virol. 76, 7760-76. (2002).
51. M. R. Wormald, et al., Chem. Rev. 102, 371-86. (2002).
52. A. J. Petrescu, M. R. Wormald, unpublished data.
53. P. D. Kwong, et al., Nature 420, 678-82. (2002).
54. W. I. Weis, K. Drickamer, Annu. Rev. Biochem. 65, 441-73 (1996).
55. T. B. Geijtenbeek, et al., Cell 100, 587-97. (2000).
56. H. Feinberg, D. A. Mitchell, K. Drickamer, W. I. Weis, Science 294, 2163-6 (2001).
57. D. A. Mitchell, A. J. Fadden, K. Drickamer, J. Biol. Chem. 276, 28939-45 (2001).
58. M. R. Boyd, et al., Antimicrob. Agents Chemother. 41, 1521-30. (1997).
59. M. T. Esser, et al., J. Virol. 73, 4360-71. (1999).
60. B. R. O'Keefe, et al., Mol. Pharmacol. 58, 982-92. (2000).
61. I. Botos, et al., J. Biol. Chem. 277, 34336-42. (2002).
62. F. Yang, et al., J. Mol. Biol. 288, 403-12. (1999).
63. L. G. Barrientos, A. M. Gronenborn, Biochem. Biophys. Res. Commun. 298, 598-602. (2002).
64. L. C. Chang, C. A. Bewley, J. Mol. Biol. 318, 1-8. (2002).
65. P. D. Kwong, et al., Structure Fold Des. 8, 1329-39. (2000).
66. G. Ragupathi, et al., Proc. Natl. Acad. Sci. 99, 13699-704. (2002).
67. R. M. Esnouf, Acta Cryst. D55, 938-40. (1999).
68. P. J. Kraulis, J. Appl. Cryst. 24, 946-950 (1991).
69. E. A. Merritt, D. J. Bacon, Meth. Enzymol. 277, 505-524 (1997).
70. M. L. Phillips, M. H. Tao, S. L. Morrison, V. N. Schumaker, Mol. Immunol. 31, 1201-10. (1994).
71. B. Carrasco, et al., Biophys. Chem. 93, 181-96. (2001).
72. A. Nicholls, K. A. Sharp, B. Honig, Proteins 11, 281-96 (1991).
73. R. A. Laskowski, MacArthur, M. W., Moss, D. S, Thornton, J. M., J. Appl. Cryst. 26, 283-291 (1993).

74. M. Dubois, et al., Anal. Chem. 28, 350-356. (1956).

TABLE 1

| | | Fab 2G12 + | Fab 2G12 + |
|---|---|---|---|
| | Fab 2G12 Unliganded | Manα1-2Man | Man₉GlcNAc₂ |
| Data Collection | | | |
| Wavelength (Å) | 0.976 | 0.984 | 0.984 |
| Resolution^{a} (Å) | 50-2.2 (2.24-2.2) | 50-1.75 (1.78-1.75) | 50-3.0 (3.05-3.0) |
| # of molecules in | 2 | 2 | 2 |
| asymmetric unit | | | |
| # of observations | 186810 | 650716 | 129852 |
| # of unique reflections | 59743 | 126361 | 29134 |
| Completeness (%) | 93.8 (85.6) | 95.6 (68.4) | 97.5 (89.9) |
| R_{sym} (%) | 6.1 (31.5) | 5.1 (40.5) | 7.2 (48.4) |
| Average I/σ | 32.9 (4.2) | 41.9 (2.4) | 27.2 (3.1) |
| Refinement statistics (all | | | |
| reflections >0.0σF) | | | |
| Resolution (Å) | 50-2.2 (2.26-2.2) | 50-1.75 (1.79-1.75) | 50-3.0 (3.08-3.0) |
| Total # of relections used | 59686 | 126118 | 29134 |
| # in test set | 3010 | 6410 | 1429 |
| R_{cryst} (%) | 22.4 (31.9) | 22.8 (42.2) | 24.8 (50.5) |
| R_{free} (%) | 26.6 (36.8) | 25.0 (43.8) | 32.9 (47.9) |
| # of Fab atoms | 6606 | 6554 | 6616 |
| # of ligand atoms | — | 35 | 261 |
| # of waters | 269 | 487 | — |
| Wilson B^{b} | 40.8 | 32.3 | 77.1 |
| B₁₁ | −11.7 | −5.2 | −34.2 |
| B₂₂ | −7.3 | −8.7 | −14.1 |
| B₃₃ | 19.0 | 13.9 | 48.2 |
| <B> values | | | |
| Variable domain 1 | 34.7 | 35.9 | 61.7 |
| Variable domain 2 | 41.6 | 40.4 | 96.9 |
| Constant domain 1 | 43.1 | 54.6 | 91.6 |
| Constant domain 2 | 49.1 | 73.8 | 118.0 |
| Ligand | — | 44.5 | 100.5 |
| Waters | 41.3 | 48.0 | — |
| Ramachandran Plot^{c} (%) | | | |
| Most favored | 90.4 | 90.3 | 68.2 |
| Additionally allowed | 8.5 | 8.9 | 26.3 |
| Generously allowed | 0.4 | 0.3 | 4.1 |
| Disallowed^{d} | 0.7 | 0.5 | 1.3 |
| R.m.s deviations | | | |
| Bond lengths (Å) | .014 | .014 | .037 |
| Angles (°) | 1.5 | 1.3 | 3.3 |

TABLE 2

| | % Affinity | | Secondary | |
|---|---|---|---|---|
| | Relative to | Primary | Interface | V_{H}/V_{H}′ |
| 2G12 Mutant | Wild Type | Combining Site | Binding Site | interface |
| H I19A | 0.5 | | | • |
| H H32A | 0.4 | • | | |
| H T33A | 2 | • | | |
| H R39A | 130 | | | |
| H R39Q | 170 | | | |
| H T52aA | 190 | • | | |
| H S54A | 0.1 | | | |
| H T55A | 0.1 | | • | |
| H Y56A | 2 | | • | |
| H R57A | 0.1 | | • | • |
| H L74A | 130 | | • | |
| H E75A | 1.1 | | • | |
| H E76A | 103 | | | |
| H F77A | 0.1 | | | • |
| H Y79A | 0.1 | | | • |
| H K82bA | 51 | | | |
| H R83A | 59 | | | |
| H V84A | 0.1 | | | |
| H K95A | 170 | • | | |
| H G96A | 20 | • | | |
| H L100A | 0.1 | • | | |
| H S100aA | 0.1 | • | | |
| H D100bA | 52 | • | | |
| H N100cA | 0.1 | • | | |
| H D100dA | 120 | • | | |
| H P113A | 0.1 | | | |
| H P113S | 0.1 | | | |
| L S28A | 0.1 | | | |
| L W32A | 50 | | | |
| L G93A | 120 | • | | |

Although the invention has been described with reference to the above example, it will be understood that modifications and variations are encompassed within the spirit and scope of the invention. Accordingly, the invention is limited only by the following claims.

1. An isolated domain-exchanged binding molecule comprising a heavy chain with a variable region and a constant region and a multivalent binding surface comprising two conventional antigen binding sites and at least one non-conventional binding site formed by an interface between adjacently positioned heavy chain variable regions; with the proviso that the molecule is not a 2G12 antibody.

2. The domain-exchanged binding molecule of claim 1, wherein the molecule is an antibody.

3. The domain-exchanged binding molecule of claim 1, wherein the domain-exchanged binding molecule has an affinity for carbohydrates.

4. The domain-exchanged binding molecule of claim 1, wherein the domain-exchanged binding molecule binds to HIV.

5. The domain-exchanged binding molecule of claim 1, wherein the domain-exchanged binding molecule binds to CD20.

6. A non-naturally occurring domain-exchanged binding molecule comprising a heavy chain with a variable region and a constant region and a multivalent binding surface comprising two conventional antigen binding sites and at least one non-conventional binding site formed by an interface between adjacently positioned heavy chain variable regions.

7. The non-naturally occurring domain-exchanged binding molecule of claim 6, wherein the molecule is an antibody.

8. The non-naturally occurring domain-exchanged binding molecule of claim 6, wherein the domain-exchanged binding molecule has an affinity for carbohydrates.

9. The non-naturally occurring domain-exchanged binding molecule of claim 6, wherein the domain-exchanged binding molecule binds to HIV.

10. The non-naturally occurring domain-exchanged binding molecule of claim 6, wherein the domain-exchanged binding molecule binds to CD20.

11. A library containing a plurality of domain-exchanged binding molecules, wherein each member of the library comprises a heavy chain with a variable region and a constant region and a multivalent binding surface comprising two conventional antigen binding sites and at least one non-conventional binding site formed by an interface between adjacently positioned heavy chain variable regions.

12. The library of claim 11, wherein the domain-exchanged binding molecules are antibodies.

13. A domain-exchanged binding molecule of claims 1 or 6, wherein the molecule is an Fab region.

14. A method of detecting infection or disease in a subject comprising:
(a) contacting a sample from a subject suspected of having an infection or disease with a domain-exchanged binding molecule of claims 1 or 6 under appropriate conditions and for sufficient time so as to allow a molecule in the sample to bind to the domain-exchanged binding molecule; and
(b) detecting the domain-exchanged binding molecule having a molecule bound thereto,
wherein a domain-exchanged binding molecule bound to a molecule is indicative of infection or disease in the subject.

15. The method of claim 14, wherein the infection or disease is an HIV-induced disease.

16. The method of claim 15, wherein the molecule is an HIV molecule.

17. The method of claim 14, wherein the infection or disease is a tumor cell.

18. The method of claim 14, wherein the infection or disease is a metastases.

19. The method of claim 14, wherein the domain-exchanged binding molecule is bound to a carrier.

20. The method of claim 14, wherein the contacting is in the presence of a blocking agent.

21. The method of claim 14, wherein the contacting is in the presence of a detectably labeled antibody having an affinity for the domain-exchanged binding molecule.

22. The method of claim 14, wherein the domain-exchanged binding molecule is coupled to a hapten.

23. The method of claim 14, wherein the domain-exchanged binding molecule is labeled with any one of enzymes, radioisotopes, fluorescent compounds, colloidal metals, chemiluminescent compounds, phosphorescent compounds, bioluminescent compounds, or combinations thereof.

24. A pharmaceutical composition comprising a domain-exchanged binding molecule of claims 1 or 6.

25. The pharmaceutical composition of claim 24, wherein the domain-exchanged binding molecule has an affinity for carbohydrates.

26. The pharmaceutical composition of claim 24, wherein the domain-exchanged binding molecule binds to HIV.

27. The pharmaceutical composition of claim 24, wherein the domain-exchanged binding molecule binds to CD20.

28. The pharmaceutical composition of claim 24, further comprising a pharmaceutically acceptable carrier.

29. A kit for determining the presence of an antigen in a sample comprising at least one domain-exchanged binding molecule of claims 1 or 6.

30. The kit of claim 29, further comprising an antibody as a separately packaged reagent, in addition to the domain-exchanged binding molecule.

31. A method of treating a subject having or at risk of having an infection or disease comprising administering to the subject a therapeutically effective amount of a domain-exchanged binding molecule of claims 1 or 6, thereby providing passive immunization to the subject.

32. The method of claim 31, wherein the infection or disease is caused by a pathogen or agent having repeating units on its surface.

33. The method of claim 31, wherein the domain-exchanged binding molecule is an antibody.

34. The method of claim 31, wherein the domain-exchanged binding molecule has an affinity for carbohydrates.

35. The method of claim 31, wherein the domain-exchanged binding molecule binds to HIV.

36. The method of claim 31, wherein the domain-exchanged binding molecule binds to CD20.
